(19) United States
(12) Patent Application Publication
(10) Pub. No.: US 2017/0260490 A1
(43) Pub. Date: Sep. 14, 2017

(54) PRIMARY MAMMALIAN CELL CULTURE SYSTEMS AND METHODS

(71) Applicant: HUREL CORPORATION, Beverly Hills, CA (US)

(72) Inventors: Erik NOVIK, EDISON, NJ (US); Amit PAREKH, Edison, NJ (US); Eric PLUDWINSKI, East Brunswick, NJ (US); Anil SHRIRAO, Edison, NJ (US)

(73) Assignee: HUREL CORPORATION, Beverly Hills, CA (US)

(21) Appl. No.: 15/504,984

(22) PCT Filed: Aug. 19, 2015
(86) PCT No.: PCT/US15/45939

§ 371 Date: Feb. 17, 2017

Related U.S. Application Data
(60) Provisional application No. 62/039,103, filed on Aug. 19, 2014.

(51) Int. Cl. C12M 1/00; C12M 1/04; C12N 5/071; C12M 1/32

(52) U.S. Cl. CPC C12M 23/52; C12M 47/20; C12M 23/12; C12M 45/22; C12M 23/24; C12M 23/26; C12M 23/38; C12N 5/067

(57) ABSTRACT

Primary mammalian cell culture systems, methods of making primary mammalian cell culture systems, and methods of culturing primary mammalian cells are provided. In some embodiments the systems and methods utilize an exothermic chemical process as a heat source for culturing and shipping cultures comprising primary mammalian cells.

### INTRODUCTION

Primary mammalian cell cultures can mimic the in vivo state and generate more physiologically relevant data than alternative systems such as cultured cell lines. However, working with primary mammalian cells can be more complex than working with cell lines. For example, lot-to-lot (i.e, donor-to-donor) variation in primary mammalian cell functional performance can occur. Also, cultures made from primary mammalian cellular materials may be less hardy and more quickly perishable, and the applicable culture methods may require more skill and delicacy, than in the case of cell lines. For these and other reasons it may be desirable to use a standardized and reproducible process to establish primary mammalian cell cultures with uniform features. For some applications it would be useful to manufacture at one location primary mammalian cell cultures having useful features, and then ship the cultures to other locations where they arrive ready for use immediately or soon after arrival. The procedures disclosed herein enable, for example, a manufacturer of primary mammalian cell cultures to manufacture the cultures at a first location and to then ship the cultures to a customer at a second location.

Prior art techniques for culturing and shipping primary mammalian cells have generally relied on cryopreservation of primary cells and shipping the cells in a cryopreserved state. Such an approach requires the recipient of the cryopreserved primary cells to then establish a primary mammalian cell culture after receipt of the cells. This approach has several potential drawbacks. First, it requires that each user have the equipment and expertise to derive a functional primary mammalian cell culture having a set of useful properties from cryopreserved primary cells. Second, it means that there is necessarily a significant time lag between receipt of the cryopreserved primary cells and the availability of a functional culture of primary mammalian cells suitable for use in an assay. This approach is also necessarily susceptible to greater variation between lots of primary cells than if a user is able to receive from a supplier cultures of primary mammalian cells having standardized structural and functional features. Shipping cells in a cryopreserved state also incurs higher expense. For these and other reasons many users of primary mammalian cell cultures will find it desirable to obtain from a supplier cell cultures having useful functional and/or morphological features, which cultures are ready to use immediately or soon after arrival.

An alternative prior art approach is to ship cells in culture so that the temperature which is optimal for the type of primary mammalian cells being shipped is maintained throughout the course of shipment. In some embodiments, the optimal temperature is the normal temperature found in the respective mammalian species in vivo, e.g., in the case of human cells, 37° C. In theory this approach could avoid the need to rehabilitate the cells after exposure to the stresses of cryopreservation, and could provide a primary mammalian cell culture to the user in a ready-to-use state. However, shipping cells in this way requires shipping not just the cells but also an incubator to maintain the cells at the their optimal temperature. There are severe logistical difficulties imposed by this approach, including the need to provide an energy source for the incubator, the weight, size, and consequent cost of shipping the incubator containing the cell cultures, the resultant economic need to recover and re-use the incubator, and the requirement for special physical handling of the incubator during the shipping process. For these and other reasons the incubator approach is prohibitively expensive and burdensome for most users.

Accordingly, there is a need in the art for new methods and systems for culturing, packaging and shipping cultures comprising primary mammalian cells that allows for maintaining the cell culture or other material during shipment without the need for either cryopreservation or for culture only at the optimal temperature for the type of cells or other material being shipped, such that the cell culture or other material is ready to use immediately or soon after receipt by a user. Such methods and systems will have many uses, including in the culture, packaging and shipping of cell cultures comprising primary mammalian cells. The invention disclosed herein meets these and other needs in the art.

### SUMMARY

This invention provides primary mammalian cell culture systems, methods of making primary mammalian cell culture systems, and methods of culturing primary mammalian cells. In some embodiments the systems and methods utilize an exothermic chemical process as a heat source for culturing and shipping cultures comprising primary mammalian cells.

This invention encompasses cell culture systems. In some embodiments the cell culture systems comprise a first container and a second container, the first container disposed within the second container; a cell culture plate disposed within the first container; and a heat source disposed inside of the second container and outside of the first container. In some embodiments the walls of the first and/or second containers are made of extruded polystyrene (EPS) foam. In some embodiments the first and second containers are dimensioned to provide a space between an inner wall of the second container and an outer wall of the first container. In some embodiments the systems further comprise at least one stabilizer comprising a first edge adjacent to an inner wall of the second container and a second edge adjacent to an outer wall of the first container. In some embodiments the heat source is positioned between the inner wall of the second container and the outer wall of the first container. In some embodiments the cell culture plate contains a culture comprising adherent primary mammalian cells. In some embodiments the adherent primary mammalian cells are hepatocytes. In some embodiments the cell culture plate comprises a base, a cover, and a layer of elastomeric material disposed between the base and cover to provide a liquid barrier to retain the culture media in the well. In some embodiments the elastomeric material is gas permeable. In some embodiments the elastomeric material is polydimethylsiloxane (PDMS). In some embodiments the heat source is an exothermic chemical process. In some embodiments the heat source comprises a battery or other source of electrical energy and means for converting that electrical energy to heat. In some embodiments the system further comprises a means of measuring changes in the temperature inside the first and/or second container. In some embodiments the system further comprises a means of increasing or decreasing the generation of heat by the heat source. In some embodiments the means of increasing or decreasing the generation of heat by the heat source is coupled to means of measuring changes in the temperature inside the first and/or second container. In some embodiments the heat source comprises a third container. In some embodiments the heat source comprises a third container comprising the substrates and/or products of an exothermic chemical process. In some embodiments the peak surface temperature of the third container is from 34° C. to 40° C. In some embodiments the first container comprises an interlocking base and lid. In some embodiments the second container (200) comprises an interlocking base and lid.

This invention also encompasses methods of making a cell culture system. In some embodiments the methods comprise A) providing a first container, a second container, a cell culture plate, and a heat source; B) establishing a primary mammalian cell culture in the cell culture plate; and C) configuring the system so that 1) the first container is disposed within the second container, 2) the cell culture plate comprising the primary mammalian cell culture is disposed within the first container, and 3) the heat source is disposed inside of the second container and outside of the first container. In some embodiments the walls of the first and/or second containers are made of extruded polystyrene (EPS) foam. In some embodiments the first and second containers are dimensioned to provide a space between an inner wall of the second container and an outer wall of the first container. In some embodiments the heat source is positioned between the inner wall of the second container and the outer wall of the first container. In some embodiments the primary mammalian cells are adhered to at least one surface of the culture plate. In some embodiments the cell culture plate comprises a base, a cover, and a layer of elastomeric material disposed between the base and cover to provide a liquid barrier to retain the culture media in the well. In some embodiments the elastomeric material is gas permeable. In some embodiments the elastomeric material is polydimethylsiloxane (PDMS). In some embodiments the heat source is an exothermic chemical process. In some embodiments the heat source comprises a battery or other source of electrical energy and means for converting that electrical energy to heat. In some embodiments the system further comprises a means of measuring changes in the temperature inside the first and/or second container. In some embodiments the system further comprises a means of increasing or decreasing the generation of heat by the heat source. In some embodiments the means of increasing or decreasing the generation of heat by the heat source is coupled to means of measuring changes in the temperature inside the first and/or second container. In some embodiments the heat source comprises a third container. In some embodiments the heat source comprises a third container comprising the substrates and/or products of an exothermic chemical process. In some embodiments the peak surface temperature of the third container is from 34° C. to 40° C.

The invention also encompasses methods of culturing cells. In some embodiments the methods comprise: A) providing a cell culture system comprising a first container, a second container, a cell culture plate comprising a primary mammalian cell culture, and a heat source, wherein the cell culture system is configured such that 1) the first container is disposed within the second container, 2) the cell culture plate comprising the primary mammalian cell culture is disposed within the first container, and 3) the heat source is disposed inside of the second container and outside of the first container; and B) maintaining the cell culture plate comprising a primary mammalian cell culture in the configured cell culture system for a first culture period. In some embodiments the walls of the first and/or second containers are made of extruded polystyrene (EPS) foam. In some embodiments the first and second containers are dimensioned to provide a space between an inner wall of the second container and an outer wall of the first container. In some embodiments the heat source is positioned between the inner wall of the second container and the outer wall of the first container. In some embodiments the primary mammalian cells are adhered to at least one surface of the culture plate. In some embodiments the cell culture plate comprises a base, a cover, and a layer of elastomeric material disposed between the base and cover to provide a liquid barrier to retain the culture media in the well. In some embodiments the elastomeric material is gas permeable. In some embodiments the elastomeric material is polydimethylsiloxane (PDMS). In some embodiments the heat source is an exothermic chemical process. In some embodiments the heat source comprises a battery or other source of electrical energy and means for converting that electrical energy to heat. In some embodiments the system further comprises a means of measuring changes in the temperature inside the first and/or second container. In some embodiments the system further comprises a means of increasing or decreasing the generation of heat by the heat source. In some embodiments the means of increasing or decreasing the generation of heat by the heat source is coupled to means of measuring changes in the temperature inside the first and/or second container. In some embodiments the heat source comprises a third container. In some embodiments the heat source comprises a third container comprising the substrates and/or products of an exothermic chemical process. In some embodiments the peak surface temperature of the third container is from 34° C. to 40° C. In some embodiments the first culture period is for from 2 to 24 hours. In some embodiments the temperature inside the first container is from 18° C. to 38° C. for the first culture period. In some embodiments the temperature inside the first container is from 28° C. to 38° C. for the first culture period. In some embodiments the temperature inside the first container is no more than 32° C. for from one to six hours during the first culture period. In some embodiments the temperature inside the first container is no more than 28° C. for from one to six hours during the first culture period. In some embodiments the difference between the minimum and maximum temperature experienced by the inside of the first container during the first culture period is from 2° C. to 20° C. In some embodiments the primary cells are primary hepatocytes.

In some embodiments the methods further comprise removing the cell culture plate from the first container after the first culture period and maintaining the cell culture plate at a temperature of about 37° C. for a second culture period of from 3 to 24 hours. In some embodiments the primary cells are primary hepatocytes, and the level of metabolic activity of the cultured primary hepatocytes after the second culture period is at least 50% of the level of metabolic activity of the cultured primary hepatocytes at initiation of the first culture period.

### BRIEF DESCRIPTION OF THE DRAWINGS

FIG. 1 shows a schematic diagram of an embodiment of a system of the invention.

FIG. 2 shows an embodiment of heat sources attached to an inner face of a vertical side wall of the outer chamber of the system.

FIG. 3 shows an embodiment of the components loaded into the inner chamber.

FIGS. 4A and 4B show an embodiment of A) a 96-well cell culture plate packing sequence, and B) a stack of packed 96-well plates secured with elastic bands.

FIG. 5A shows a graph of temperature over time inside the inner chamber of an embodiment of a system of the invention during a shipment from New Jersey to California that comprised an air travel segment. The inner chamber also included a culture of primary human hepatocytes.

FIG. 5B demonstrates the CYP 3A4 activity of the primary human hepatocytes. The CYP 3A4 activity of control cells maintained in an incubator (constant 37° C. and 5% CO₂) are marked “incubator”. The CYP 3A4 activity of control cells maintained at a constant 37° C. without CO₂ regulation) are marked “37 C”. The CYP 3A4 activity of cells shipped and then incubated at constant 37° C. and 5% CO₂ at the destination are marked “destination”. The results show that shipped cells that are then cultured at a constant 37° C. with CO₂ regulation have a stable enzymatic activity after arrival at the shipping destination.

FIG. 6A shows a graph of temperature over time inside the inner chamber of a system of the invention during a shipment from New Jersey to France that comprised an air travel segment. After arrival in France the system was delivered to a location in the same metropolitan area as the destination airport. The inner chamber also included a culture of primary human hepatocytes.

FIG. 6B shows a graph of temperature over time inside the inner chamber of a system of the invention during a shipment from New Jersey to France that comprised an air travel segment followed by a segment of travel by train, followed by a segment of travel by car from the train station to the final destination. The inner chamber also included a culture of primary human hepatocytes.

FIG. 6C demonstrates the CYP 3A4 activity of the primary human hepatocytes. The CYP 3A4 activity of control cells maintained in an incubator (constant 37° C. and 5% CO₂) are marked “origin”. The CYP 3A4 activity of the cells shipped under the conditions illustrated in FIG. 6A and then incubated at constant 37° C. and 5% CO₂ at the destination are marked “intermediate”. The CYP 3A4 activity of the cells shipped under the conditions illustrated in FIG. 6B and then incubated at constant 37° C. and 5% CO₂ at the final destination are marked “destination”. The results show that shipped cells that are then cultured at a constant 37° C. with CO₂ regulation have a stable enzymatic activity after arrival at the shipping destination.

FIGS. 7A to 7C show graphs of temperature over time inside the inner chamber of three systems of the invention during shipments from New Jersey to Illinois that comprised air travel segments. Each shipment occurred on a different day.

FIGS. 8A and 8B show graphs of temperature over time inside the inner chamber of a system comprising a heat source coupled to a battery.

FIG. 9 shows a graph of temperature over time inside the inner chamber of a system configured without a heat source during a shipment from New Jersey to California that comprised an air travel segment.

### DETAILED DESCRIPTION

### A. Introduction

The inventors have discovered systems and methods for culturing primary mammalian cells without the use of either cryopreservation or an incubator powered by an external source of electric current. The systems and methods are useful, for example, to ship cultures comprising adherent primary mammalian cells from an origin to a destination. This invention is based in part on the inventors' discovery that cultures comprising adherent primary mammalian hepatocytes can be maintained at a culture temperature of less than 37° C. and under conditions characterized by culture temperature fluctuation during a first culture period while remaining adhered to a culture substrate. This finding in part enables the culture methods of the invention. The inventors have also discovered culture systems that provide for culturing adherent primary mammalian hepatocytes at a culture temperature of less than 37° C. and under conditions characterized by culture temperature fluctuation during a first culture period during which cells remain adhered to a culture substrate. These and other aspects of the invention are provided herein.

Traditional cell culture incubators powered by electric current are designed and function to maintain a constant cell culture temperature. Standard laboratory incubators used for culturing mammalian cells maintain a culture temperature to within 0.2° C. Standard practice when culturing mammalian cells is to maintain the cells at the optimal culture temperate for each cultured cell type throughout the culture period. The inventors proceeded contrary to both of these standard practices in developing the invention.

The systems of the invention are based on the novel combination of several forms of temperature buffering to provide a cell culture environment. The inventors have discovered that systems utilizing a container within a container design may be used to culture primary mammalian cells for periods of from 2 to 24 hours or longer. The systems comprise an inner container enclosing a cell culture plate comprising a primary mammalian cell culture and an outer container enclosing the inner container. A heat source is optionally located between the inner and outer containers; that is, the heat source is located outside of the inner container and inside of the outer container. The heat source is not functionally connected to an energy source outside of the outer container. This is one of several features that distinguish embodiments of the systems of the invention from traditional cell culture incubators powered by an external electric current source.

Locating a heat source between the inner and outer containers in this fashion enhances portability of several embodiments of the systems of the invention compared to traditional cell culture incubators. The heat source also provides sufficient temperature control to the cell culture plate to maintain the primary mammalian cells in culture adhered to a culture substrate.

Through use of systems of the invention the inventors have also discovered methods of culturing primary mammalian cells. In some embodiments the methods comprise providing a cell culture system of the invention comprising a cell culture plate comprising a culture comprising adherent primary mammalian cells and maintaining the cell culture system under conditions in which the temperature inside the first container is in a range of from 18° C. to 38° C. for a first culture period. The inventors have discovered that such methods provide, at the end of the first culture period, a culture comprising adherent primary mammalian cells. The culture comprising adherent primary mammalian cells is ready to use within hours in assays that rely on primary mammalian cell function.

Alternative methods of culturing primary mammalian cells are also provided that do not utilize a cell culture system of the invention. In part through the use of cell culture systems of the invention the inventors have discovered that the relationship between cell culture temperature and the functional state of cultured primary mammalian cells is less restrictive than generally appreciated in the art. In particular, the inventors have discovered that a primary mammalian cell culture may be maintained under conditions in which the culture temperature is in a range of from 18° C. to 38° C. for a first culture period to provide, at the end of the first culture period, a culture comprising adherent primary mammalian cells.

### B. Primary Mammalian Cell Culture Systems

This invention encompasses cell culture systems. The systems may be used in any context in which it is useful to culture primary mammalian cells without the use of an incubator functionally linked to an external source of electric current. One such application is in shipping a culture comprising primary mammalian cells from a source to a destination, for example when a manufacturer desires to ship a culture comprising primary mammalian cells to a customer.

In some embodiments the cell culture systems comprise a first container and a second container, the first container disposed within the second container; a cell culture plate disposed within the first container; and a heat source disposed inside of the second container and outside of the first container. In some embodiments the walls of the first and/or second containers are made of extruded polystyrene (EPS) foam. In some embodiments the first and second containers are dimensioned to provide a space between an inner wall of the second container and an outer wall of the first container. In some embodiments the systems further comprise at least one stabilizer comprising a first edge adjacent to an inner wall of the second container and a second edge adjacent to an outer wall of the first container. In some embodiments the heat source is positioned between the inner wall of the second container and the outer wall of the first container. In some embodiments the cell culture plate contains a culture comprising adherent primary mammalian cells. In some embodiments the adherent primary mammalian cells are hepatocytes. In some embodiments the cell culture plate comprises a base, a cover, and a layer of elastomeric material disposed between the base and cover to provide a liquid barrier to retain the culture media in the well. In some embodiments the elastomeric material is gas permeable. In some embodiments the elastomeric material is polydimethylsiloxane (PDMS). In some embodiments the heat source is an exothermic chemical process. In some embodiments the heat source comprises a third container. In some embodiments the heat source comprises a third container comprising the substrates and/or products of an exothermic chemical process. In some embodiments the peak surface temperature of the third container is from 34° C. to 40° C. In some embodiments the first container comprises an interlocking base and lid. In some embodiments the second container (200) comprises an interlocking base and lid.

First Container:

The first container is generally made from a material characterized by useful properties comprising at least one of thermal insulation properties, low weight, chemical inertness, shock resistance and ease of manufacturing. For example, the first container may be made from at least one material selected from Expanded Polystyrene (EPS, Styrofoam), Polyurethane (PUR), Polyethylene Foam and glass wool. In some embodiments the first container is made of Extruded Polystyrene (EPS) foam. In some embodiments the first and second containers are made of the same material. In some embodiments the first and second containers are made of different materials. In some embodiments the first and/or second containers are made of more than one material. The inside dimensions of the first container are chosen such that the first container can fit within the second container, optionally providing for a space between the outer wall of the first container and the inner wall of the second container, and also so that the first container can accommodate at least one cell culture plate inside the first container. The thickness of the walls of the first container is typically from 0.5 to 3 inches, such as from 1 to 2 inches. In some embodiments the walls of the second container are 1.5 inches thick. In some embodiments the thickness of the walls of the first and second containers are the same. In some embodiments the thickness of the walls of the first and second containers are different. In some embodiments either or both of the containers comprises walls of different thickness. The internal length, width, and height of the first container will generally independently be from 4 to 30 inches. In some embodiments the internal dimensions of the first container are independently from 5 to 9 inches. In some embodiments the dimensions of the first and second containers provide for a space between the first and second containers when the second container is placed inside the first. The air gap acts as a thermal buffer and facilitates functioning of the optional heat sources and circulation of heat generated by the heat sources in embodiments that utilize heat sources. The purpose of the first container is also two fold. First, it isolates the interior of the first container from the second container to limit the rate of temperature variation over time inside the first container. Second, the interior provides a semi-controlled environment for maintaining a cell culture plate comprising a culture comprising primary mammalian cells over a culture period. The first container may comprise upper and lower portions which interlock. Whether the upper and lower portions which interlock or not, the upper and lower portions may be secured in contact with each other using any suitable material, such as tape. This arrangement allows cell culture plate comprising a culture comprising primary mammalian cells to be placed inside the first container. In some embodiments the brims of the lower portion and the upper portion of the second container have a matched locking groove structure which not only provides tight sealing but also reduces heat transfer between the first and second containers.

Second Container:

The Second container is generally made from a material characterized by useful properties comprising at least one of thermal insulation properties, low weight, chemical inertness, shock resistance and ease of manufacturing. For example, the first container may be made from at least one material selected from Expanded Polystyrene (EPS, Styrofoam), Polyurethane (PUR), Polyethylene Foam and glass wool. In some embodiments the second container is made of Extruded Polystyrene (EPS) foam. The thickness of the walls of the second container are typically from 0.5 to 3 inches, such as from 1 to 2 inches. In some embodiments the walls of the second container are about 1.5 inches thick. The inside dimensions of the second container are chosen to accommodate the first container and with a view toward minimizing the size of the second container to aid in use of the system to transport a culture comprising primary mammalian cells by airplane, truck, and/or train. The internal length, width, and height of the second container will generally independently be from 8 to 36 inches. In some embodiments the internal dimensions of the second container are independently from 10 to 15 inches. The purpose of the second container is at least two fold. First, it partially isolates the inside of the container from ambient conditions outside the container and buffers the effect of ambient conditions on the temperature inside the container over the time course of a culture period. Second, it retains heat generated by optional heat sources inside the second container. The second container may comprise upper and lower portions which interlock. Whether the upper and lower portions interlock or not, the upper and lower portions may be secured in contact with each other using any suitable material, such as tape or elastic bands. This arrangement allows a first container to be placed inside the second container. In some embodiments the brims of the lower portion and the upper portion have a matched locking groove structure which not only provides tight sealing but also reduces the loss of heat from the second container.

Stabilizers:

The container inside container configuration in conjunction with the optional use of heat sources maintains the internal temperature of the inner, first container at from 12° C. to 38° C. over a culture period, such as at from 18° C. to 38° C. Stabilizers may be used to fix the location of the first container inside the second container. The stabilizer(s) are generally made from a material characterized by useful properties comprising at least one of thermal insulation properties, low weight, chemical inertness, shock resistance and ease of manufacturing. For example, the first container may be made from at least one material selected from Expanded Polystyrene (EPS, Styrofoam), Polyurethane (PUR), Polyethylene Foam and glass wool. This forms gap between the outer surface of the first container and the inner surface of the second container. The gap is typically filled with air but may be filled with any material, such as gas of a determined composition different than air. Gas (e.g., air) present inside the gap facilitates functioning of the heat sources. The stabilizers decrease the volume of the gap between the two containers, affecting both peak temperature of the heat sources and the duration of time over which they provide a rated temperature. The stabilizers are carefully designed to fix the inner chamber in place without significantly reducing the air gap between the two chambers while limiting the surface area of the outer surface of the inner first container that is in contact with the stabilizer, thus maximizing transfer of heat from the heat packs to the inside of the first container in a uniform fashion. The dimensions of the stabilizers are chosen in conjunction with the dimensions of the first and second containers and may vary over a large range. Typically 1, 2, 3, 4, 5, 6, 7, 8, or more stabilizers are used in a system.

Heat Sources:

The systems of the invention optionally comprise a heat source. In some embodiments the heat source comprises an exothermic chemical reaction. In some embodiments the heat source consists of an exothermic chemical reaction. The heat source provides heat energy to maintain the temperature of the cell culture plate at from 12° C. to 38° C. during a culture period, such as at from 18° C. to 38° C.

In some embodiments the system comprises at least one heat source selected from an exothermic chemical reaction; a phase change material which absorbs heat and releases the heat slowly upon change in phase, i.e. a gel pack; and an electrical heating element connected to a battery.

The exothermic chemical reaction may be any chemical process that releases heat. Several suitable examples are known. In a first type, air mediated exothermic oxidation produces heat. In some embodiments iron, cellulose, or magnesium oxide is used as the reducing agent. In one embodiment the exothermic chemical reaction occurs in a composition comprising iron, water, activated carbon (evenly distributes heat), vermiculite (water reservoir) and salt (catalyst), which produces heat from the exothermic oxidation of iron when exposed to air. In a second example the exothermic crystallization of supersaturated solutions (for example sodium acetate) provides heat. This second type can be recharged by immersing a container comprising the solution in very hot water until the contents are uniformly fluid and then allowing it to cool. The release of heat is triggered by flexing a small metal disk inside the container, which generates nucleation centers that initiate crystallization. Heat is required to dissolve the salt in its own water of crystallization and it is this heat that is released when crystallization is initiated.

In some embodiments the heat source comprises a container. In some embodiments the heat source comprises a container comprising the substrates and/or products of an exothermic chemical process. In some embodiments the peak surface temperature of the container is from 34° C. to 45° C., from 35° C. to 44° C., from 36° C. to 43° C., from 37° C. to 42° C., from 38° C. to 46° C., from 40° C. to 44° C., from 41° C. to 43° C., or about 42° C. In some embodiments the surface temperature of the heat source maintains a surface temperature that is within 5° C. of the peak surface temperature of the heat source for from 2 to 24 hours, from 2 to 20 hours, from 2 to 16 hours, from 2 to 12 hours, from 2 to 8 hours, from 2 to 4 hours, from 4 to 24 hours, from 4 to 20 hours, from 4 to 16 hours, from 4 to 12 hours, from 4 to 8 hours, from 8 to 24 hours, from 8 to 20 hours, from 8 to 16 hours, or from 8 to 12 hours when incubated at 28° C., 24° C., 20° C., 16° C., 12° C., 8° C., or 4° C. In some embodiments the surface temperature of the heat source maintains a surface temperature that is within 5° C. of the peak surface temperature of the heat source for at least 2 hours, 4 hours, 6 hours, 8 hours, 10 hours, 12 hours, 14 hours, 16 hours, 18 hours, 20 hours, 22 hours, or 24 hours when incubated at 28° C., 24° C., 20° C., 16° C., 12° C., 8° C., or 4° C.

The heat source may be mounted on an interior surface of a wall of the second container, for example using a pouch secured to the inner wall. The heat source may be mounted on an exterior surface of a wall of the first container, for example using a pouch secured to the exterior wall. In some embodiments the heat source is mounted on a surface of the wall that is flush with adjacent portions of the wall. In some embodiments the heat source is mounted in a recessed location of the wall. As used herein, a “wall” of the container may be a lateral, top, or bottom portion of the container when the container is oriented for use.

Locating the heat source outside of the first container and inside of the second container enables some of the heat released from the heat source to penetrate a wall of the first container by conduction. Some of the heat released by the heat source is retained for a time in the area between the two containers. This configuration thus prevents the direct propagation of heat waves from the heat packs to the interior of the first container. Therefore, the peak temperature reached in the interior of the first container is lower than the peak temperature reached adjacent to the heat source in the area between the two containers. The number of heat sources required to maintain the temperature inside first container at a temperature range, such as from 12° C. to 38° C. or such as from 12° C. to 38° C. depends on the dimensions of the two chambers and the dimensions of the area between two chambers. Several factors while impact the choice of the type of heat source and the number of heat sources to incorporated into the system. For example, if the system will be used to maintain a culture comprising primary mammalian cells for a culture period the length of the culture period may influence the type and/or number of heat sources. If the culture period will be shorter (e.g., 6 hours) a single heat source may be chosen. If the culture period will be longer (e.g., 18 hours) a plurality (e.g., 4) heat sources may be chosen. As the peak surface temperature of the heat source is higher it may be desirable to increase the thickness of a wall of the first container in contact with the heat source, whereas if the peak surface temperature of the heat source is lower it may be desirable to decrease the thickness of a wall of the first container in contact with the heat source. In some embodiments the system comprises a plurality of different types of heat source.

In some embodiments initiation of heat production by a heat source is controlled and occurs after a first culture period begins. For example, the heat source may be coupled to a thermostat that initiates heat production only when/if the temperature inside the first and/or second container drops below a certain threshold. In some embodiments that utilize an exothermic chemical reaction as a heat source, initiation of the exothermic chemical reaction may be controlled in this manner.

In some embodiments that utilize an exothermic chemical reaction as a heat source, the rate of production of heat may change over time. In some embodiments the rate of production of heat by the heat source is modulated by addition of further components to the chemical reaction, and/or the times release of substrates of the chemical reaction.

In some embodiments the heat source comprises a battery or other source of electrical energy and means for converting that electrical energy to heat. In some embodiments the system comprises a) a heat source comprising an exothermic chemical reaction; and b) a heat source comprising a battery or other source of electrical energy and means for converting that electrical energy to heat. In some embodiments a single heat source comprises both an exothermic chemical reaction; and a battery or other source of electrical energy and means for converting that electrical energy to heat. For example, the battery or other source of electrical energy and means for converting that electrical energy to heat may be configured to regulate the rate of formation of heat by the exothermic chemical reaction.

In some embodiments the system further comprises a means of measuring changes in the temperature inside the first and/or second container. For example, the system may comprise a thermometer coupled to a switch. In some embodiments the system further comprises a means of increasing or decreasing the generation of heat by the heat source. In some embodiments the means of increasing or decreasing the generation of heat by the heat source is coupled to means of measuring changes in the temperature inside the first and/or second container.

Cell Culture Plates:

At least one cell culture plate is placed inside the second container. The cell culture plate comprises at least one well comprising a culture comprising primary mammalian cells. The culture may be any culture described in this disclosure. In some embodiments the primary mammalian cells in the culture are adhered to a surface of the at least one well. In some embodiments the culture plate further comprises a layer of elastomeric material disposed between the lower and upper portions of the culture plate, wherein the elastomeric layer provides a liquid barrier to retain the culture media in the well. In some embodiments the elastomeric material is gas permeable. In some embodiments the elastomeric material is polydimethylsiloxane (PDMS). In some embodiments the elastomeric material is selected from bio-compatible rubber sheets, PMMA, Teflon, and Silicone Rubber.

Thermal Properties of the Systems:

The thermal properties of the system may be characterized by exposing the system to an environment comprising an ambient temperature for a period of time. In some embodiments of the system the cell culture plate that is initially at 37° C. is maintained at a temperature of from 12° C. to 35° C., from 12° C. to 30° C., from 12° C. to 25° C., from 12° C. to 20° C., from 12° C. to 15° C., from 15° C. to 35° C., from 15° C. to 30° C., from 15° C. to 25° C., from 15° C. to 20° C., from 20° C. to 35° C., from 20° C. to 30° C., from 20° C. to 25° C., from 25° C. to 35° C., from 25° C. to 30° C., or from 30° C. to 35° C. when the system is maintained in an environment comprising an ambient temperature of 4° C. for 10, 15, 20, 25 or 30 hours.

In some embodiments of the system the cell culture plate that is initially at 37° C. is maintained at a temperature of no more than 35° C., no more than 34° C., no more than 32° C., no more than 30° C., no more than 28° C., no more than 26° C., no more than 24° C., no more than 22° C., no more than 20° C., no more than 18° C., no more than 16° C., no more than 14° C., or no more than 12° C., when the system is maintained in an environment comprising an ambient temperature of 4° C. for 10, 15, 20, 25 or 30 hours.

In some embodiments of the system the cell culture plate that is initially at 37° C. is maintained at a range of temperature of at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. during the first culture period. In some embodiments the temperature of the cell culture varies over a range of from 2° C. to 16° C., from 2° C. to 14° C., from 2° C. to 12° C., from 2° C. to 10° C., from 2° C. to 8° C., from 2° C. to 6° C., from 2° C. to 4° C., 4° C. to 16° C., from 4° C. to 14° C., from 4° C. to 12° C., from 4° C. to 10° C., from 4° C. to 8° C., from 4° C. to 6° C., 6° C. to 16° C., from 6° C. to 14° C., from 6° C. to 12° C., from 6° C. to 10° C., from 6° C. to 8° C., 8° C. to 16° C., from 8° C. to 14° C., from 8° C. to 12° C., from 8° C. to 10° C., 10° C. to 16° C., from 10° C. to 14° C., from 10° C. to 12° C., 12° C. to 16° C., or from 12° C. to 14° C. when the system is maintained in an environment comprising an ambient temperature of 4° C. for 10, 15, 20, 25 or 30 hours.

The thermal properties of the systems contribute to the usefulness of the systems for transporting cultures comprising primary mammalian cells. For example, the thermal properties of the systems enabled maintaining adherent cultures comprising primary mammalian cells during shipping by air travel.

### C. Cultures Comprising Primary Mammalian Cells

As used herein, a “primary cell” is a cell derived directly from a subject. Primary cells have a limited lifespan. After a certain number of population doublings (called the Hayflick limit), cells undergo the process of senescence and stop dividing, while generally retaining viability. Primary cells can be contrasted with a stablished or immortalized cell line, which has acquired the ability to proliferate indefinitely either through random mutation or deliberate modification, such as artificial expression of the telomerase gene.

The systems of the invention comprise a culture of primary mammalian cells. Numerous methods of isolating primary mammalian cells are known in the art and skilled artisans are able to apply well known techniques to isolate and culture new primary mammalian cell types. Accordingly, the primary mammalian cells used in the invention may be any type of primary mammalian cell. Primary mammalian cells of different types are also available from vendors, such as LIFE TECHNOLOGIES CORPORATION. Examples of primary cell types which may be purchased from LIFE TECHNOLOGIES CORPORATION include corneal epithelial cells, fibroblasts, hepatocytes, keratinocytes, mammary epithelial cells, melanocytes, microvascular endothelial cells, large vessel endothelial cells, neuronal cells, glial cells, neural stem cells, skeletal myoblasts, and smooth muscle cells. In some embodiments the primary cells are provided in cryopreserved form, and are then thawed prior to seeding to establish a culture comprising primary mammalian cells.

The primary mammalian cells may be from any mammal, including without limitation human, non-human primate (such as a cynomolgus monkey), farm animal (such as pig, horse, cow, and sheep), a domestic mammal (such as dogs, cats, guinea pig and rabbit), and rodents (such as mice and rats).

In some embodiments the primary cells are hepatocytes. Primary hepatocytes used to establish the cultures may but need not be supplied in cryopreserved form. Cropreserved human hepatocytes may be obtained from LIFE TECHNOLOGIES CORPORATION. Cropreserved non-human primate hepatocytes may be obtained from LIFE TECHNOLOGIES CORPORATION. Cropreserved dog hepatocytes may be obtained from IVT BIORECLAMATION. Cropreserved rat hepatocytes may be obtained from LIFE TECHNOLOGIES CORPORATION. In some embodiments the primary hepatocytes used to establish the culture are freshly isolated and have not been cryopreserved following isolation.

In some embodiments the culture of primary mammalian cells comprises only a single type of primary mammalian cell. In some embodiments the culture of primary mammalian cells comprises a plurality of primary mammalian cell types. In some embodiments the culture of primary mammalian cells consists of only primary mammalian cells.

In some embodiments the culture of primary mammalian cells comprises primary mammalian cells and at least one non-primary cell type. In some embodiments the at least one non-primary cell type is a cell line. In some embodiments the at least one non-primary cell type is a stromal cell. In some embodiments the at least one non-primary cell type is a non-stromal cell. In some embodiments the at least one non-primary cell type is a parenchymal cell. In some embodiments the at least one non-primary cell type is a non-parenchymal cell.

In some embodiments the culture comprises primary cells from more than one type of mammal together in a single culture. For example, primary cells from a human and from a dog. In some embodiments all of the primary cells in the culture are from the same type of mammal, for example a human.

In some embodiments the culture comprises at least one non-primary cell type, and the at least one non-primary cell type is from the same type of mammal as at least one type of primary cells in the culture. In some embodiments the culture comprises at least one non-primary cell type, and the at least one non-primary cell type is from a different type of mammal than the primary cells in the culture. In some embodiments all of the cells in the culture are from the same type of mammal.

In some embodiments the culture is a co-culture comprising at least one type of primary mammalian cell and at least one type of non-primary mammalian cell. In some embodiments of the coculture a single type of primary cell and a single type of non-primary cell represent at least 95%, at least 96%, at least 97%, at least 98%, at least 99%, at least 99.5%, at least 99.9%, or at least 99.99% of the cells in the coculture. Typically the primary cells and the non-primary cells are present in the coculture at a ratio of from 1:10 to 10:1. In some embodiments the primary cells and the non-primary cells are present in the coculture at a ratio of from 2:10 to 10:2. In some embodiments the primary cells and the non-primary cells are present in the coculture at a ratio of from 2:10 to 4:10. In some embodiments the primary cells and the non-primary cells are present in the coculture at a ratio of from 4:10 to 6:10. In some embodiments the primary cells and the non-primary cells are present in the coculture at a ratio of from 6:10 to 8:10. In some embodiments the primary cells and the non-primary cells are present in the coculture at a ratio of from 8:10 to 1:1. In some embodiments the primary cells and the non-primary cells are present in the coculture at a ratio of from 1:1 to 10:8. In some embodiments the primary cells and the non-primary cells are present in the coculture at a ratio of from 10:8 to 10:6. In some embodiments the primary cells and the non-primary cells are present in the coculture at a ratio of from 10:6 to 10:4. In some embodiments the primary cells and the non-primary cells are present in the coculture at a ratio of from 10:4 to 10:2. In some embodiments the primary cells and the non-primary cells are present in the coculture at a ratio of 10:1, 10:2, 10:3, 10:4, 10:5, :10:6, 10:7, 10:8, 10:9, 1:1, 9:10, 8:10, 7:10, 6:10, 5:10, 4:10, 3:10, 2:10, or 1:10.

In some embodiments the primary cell/non-primary cell coculture comprises at least two primary cell types. In some embodiments the primary cells in the coculture comprises two primary cell types that each represent at least 0.01%, at least 0.1%, at least 0.5%, at least 1%, at least 2%, at least 3%, at least 4%, at least 5%, or at least 10% of the cells in the coculture.

In some embodiments the primary cell/non-primary cell coculture comprises at least two non-primary cell types. In some embodiments the primary cell/non-primary cell coculture comprises two non-primary cell types that each represent at least 0.01%, at least 0.1%, at least 0.5%, at least 1%, at least 2%, at least 3%, at least 4%, at least 5%, or at least 10% of the cells in the coculture.

Generally the primary cells in the culture are adhered to a surface of the culture substrate. For example, the primary cells may be adhered to the bottom of at least one well of a culture plate. Generally, in the case of a coculture the nonprimary cells in the coculture are also adhered to the surface of the culture substrate. For example, the nonprimary cells may be adhered to the bottom of at least one well of a culture plate. For avoidance of doubt, a first cell is adhered to a surface of a culture substrate if the first cell directly contacts and attaches to the surface or if the first cell directly contacts and attaches to a second cell and the second cell directly contacts and attaches to the surface. All such cells are adhered to the surface and are considered adherent cells as used herein.

In some embodiments the culture comprises primary hepatocytes. In some embodiments the culture is a co-culture comprising primary hepatocytes and non-parenchymal cells. In some embodiments the non-parenchymal cells are stromal cells. In some embodiments the primary hepatocytes and the stromal cells are disposed on a surface of a solid substrate. In some embodiments the hepatocytes are substantially dispursed across the surface of the solid substrate. In some embodiments the culture is a co-culture comprising two cell types wherein one cell type is primary hepatocytes and the other cell type is Kupffer cells. In some embodiments the culture is a co-culture comprising two cell types wherein one cell type is primary hepatocytes and the other cell type is a different cell type. In some embodiments the culture is a co-culture comprising three different cell types. In some embodiments the culture is a co-culture comprising more than three different cell types. In some embodiments the culture comprises a tissue slice. In some embodiments the culture comprises a cellular component or cellular material.

In some embodiments of the hepatocyte-stromal cell coculture the hepatocytes and a single stromal cell type represent at least 95%, at least 96%, at least 97%, at least 98%, at least 99%, at least 99.5%, at least 99.9%, or at least 99.99% of the cells in the coculture. Typically the hepatocytes and stromal cells are present in the coculture at a ratio of from 1:10 to 10:1. In some embodiments the hepatocytes and stromal cells are present in the coculture at a ratio of from 2:10 to 10:2. In some embodiments the hepatocytes and stromal cells are present in the coculture at a ratio of from 2:10 to 4:10. In some embodiments the hepatocytes and stromal cells are present in the coculture at a ratio of from 4:10 to 6:10. In some embodiments the hepatocytes and stromal cells are present in the coculture at a ratio of from 6:10 to 8:10. In some embodiments the hepatocytes and stromal cells are present in the coculture at a ratio of from 8:10 to 1:1. In some embodiments the hepatocytes and stromal cells are present in the coculture at a ratio of from 1:1 to 10:8. In some embodiments the hepatocytes and stromal cells are present in the coculture at a ratio of from 10:8 to 10:6. In some embodiments the hepatocytes and stromal cells are present in the coculture at a ratio of from 10:6 to 10:4. In some embodiments the hepatocytes and stromal cells are present in the coculture at a ratio of from 10:4 to 10:2. In some embodiments the hepatocytes and stromal cells are present in the coculture at a ratio of 10:1, 10:2, 10:3, 10:4, 10:5, :10:6, 10:7, 10:8, 10:9, 1:1, 9:10, 8:10, 7:10, 6:10, 5:10, 4:10, 3:10, 2:10, or 1:10.

In some embodiments the hepatocyte-stromal cell coculture comprises at least two stromal cell types. In some embodiments the hepatocyte-stromal cell coculture comprises two stromal cell types that each represent at least 0.01%, at least 0.1%, at least 0.5%, at least 1%, at least 2%, at least 3%, at least 4%, at least 5%, or at least 10% of the cells in the coculture.

In some embodiments the stromal cell type is from the same type of mammal as the hepatocyte. In some embodiments the stromal cell type is from a different type of mammal than the hepatocyte.

In some embodiments the hepatocyte-stromal cell coculture comprises a third cell type. In some embodiments the third cell type is a stromal cell. In some embodiments the third cell type is not a stromal cell. In some embodiments the third cell type is a parenchymal cell. In some embodiments the third cell type is not a non-parenchymal cell. In some embodiments the third cell type is selected from Ito cells, endothelial cells, biliary duct cells, immune-mediating cells, and stem cells. In some embodiments, the immune-mediating cells are selected from macrophages, T cells, neutrophils, dendritic cells, mast cells, eosinophils and basophils.

In some embodiments the third cell type is a Kueppfer cell. In some embodiments the Kueppfer cells represent at least 0.01%, at least 0.1%, at least 0.5%, at least 1%, at least 2%, at least 3%, at least 4%, at least 5%, or at least 10% of the cells in the coculture.

In some embodiments the stromal cell type is an endothelial cell. In some embodiments the stromal cell type is a fibroblast cell. In some embodiments the stromal cell is a primary cell. In some embodiments the stromal cell is obtained from a cell line. In some embodiments the stromal cell is a transformed cell. In some embodiments the stromal cell is differentiated in vitro from a stem cell, such as an embryonic stem cell, adult stem cell, or induced pluripotent stem cell. Numerous sources of stromal cells such as fibroblasts are known in the art and may be utilized in the hepatocyte-stromal cell cocultures. One example is the NIH 3T3-J2 cell line. (See for example US 2013/0266939 A1.).

The art teaches that some aspects of hepatocyte function in culture are improved by disposing hepatocytes and stromal cells onto a solid substrate such that the hepatocytes are attached to the substrate in a first step in a cellular island configuration. (See US 2013/0266939 A1.) Specifically, such methods rely on formation of cellular islands of hepatocytes on a substrate, the hepatocyte islands surrounded by a non-parenchymal cell type such as a stromal cell type. The hepatocyte islands are formed by first placing an extracellular matrix component or derivative onto a solid substrate in an island pattern and then allowing the hepatocytes to adhere to the extracellular matrix component or derivative. The non-parenchymal cell type is then added and allowed to “fill in” the portions of the substrate that don't contain hepatocytes. A fundamental feature of such systems is that the hepatocytes are not dispursed across the substrate surface.

In some embodiments of the hepatocyte-stromal cell coculture utilized in the systems and methods this invention, hepatocytes are distributed in a cellular island configuration such as described in US 2013/0266939 A1. However, in other embodiments the hepatocytes are substantially dispursed across the surface of the solid substrate.

As used herein, “dispersed across the surface” in reference to an arrangement of hepatocytes on a solid support in a hepatocyte-stromal cell coculture means that at least one of the following criteria applies to the coculture: 1) at least 20%, at least 30%, at least 40%, or at least 50% of the surface of the solid substrate is covered by at least one hepatocyte; 2) at least 2%, at least 5% or at least 10% of the hepatocytes in the coculture are located on top of a stromal cell that is in contact with the solid substrate; and 3) the hepatocytes were not seeded onto the solid substrate by adding the hepatocytes to a solid substrate comprising islands of at least one extracellular matrix component to create islands of hepatocytes attached to the solid substrate. Note that a single hepatocyte may be counted as meeting criteria 1 and criteria 2.

Generally, a substantial proportion of the primary cells in a culture or co-culture are adhered to a culture substrate. Maintenance of adherence reflects that the functional state and health of the cells is intact. In some embodiments the primary cells are hepatocytes. In some embodiments the culture is a hepatocyte-stromal cell coculture.

In some embodiments that utilize primary mammalian hepatocytes as a primary mammalian cell type in a culture the metabolic function of the hepatocyte-stromal cell coculture is determined by measuring an activity selected from gene expression, cell function, metabolic activity, morphology, and a combination thereof, of the hepatocytes in the coculture. In some embodiments the metabolic function of the hepatocyte-stromal cell coculture is determined by measuring the level of expression and/or activity of at least one CYP450 enzyme. The level of expression and/or activity of at least one CYP450 enzyme may be measured by measuring expression of the CYP450 enzyme mRNA, by measuring expression of the CYP450 enzyme protein, or by a functional assay of CYP450 enzyme activity. In some embodiments, the metabolic activity is a CYP450 enzyme activity. In some embodiments, the CYP450 enzyme is a CYP450 enzyme selected from CYP1A2, CYP1B1, CYP2A6, CYP2B6, CYP2C, CYP2D6, CYP2E1, CYP2F1, CYP2J2, CYP3A4, CYP4A, and CYP4B. In some embodiments the at least one CYP450 enzyme is one, two, three or all four of CYP1A2, CYP1B1, CYP2A6, and CYP3A4.

In some embodiments the metabolic function of primary hepatocytes in culture endures for at least seven days. In some embodiments the metabolic function of primary hepatocytes in culture endures for at least fourteen days. In some embodiments the metabolic function of primary hepatocytes in culture endures for at least twenty-one days. In some embodiments the metabolic function of primary hepatocytes in culture endures for at least twenty-eight days.

In some embodiments the culture comprises a tissue slice. In some embodiments the tissue slice comprises the primary mammalian cells.

In some embodiments the culture comprising primary mammalian cells is cultured in serum-free or essentially serum-free media. In some embodiments the culture comprising primary mammalian cells is cultured in media containing serum. In some embodiments the media comprises 0.1% serum, 0.2% serum, 0.3% serum, 0.4% serum, 0.5% serum, 0.6% serum, 0.7% serum, 0.8% serum, 0.9% serum, 1% serum, 2% serum, 3% serum, 4% serum, 5% serum, 6% serum, 7% serum, 8% serum, 9% serum, or 10% serum. In some embodiments the media comprises at least 0.1% serum, at least 0.2% serum, at least 0.3% serum, at least 0.4% serum, at least 0.5% serum, at least 0.6% serum, at least 0.7% serum, at least 0.8% serum, at least 0.9% serum, at least 1% serum, at least 2% serum, at least 3% serum, at least 4% serum, at least 5% serum, at least 6% serum, at least 7% serum, at least 8% serum, at least 9% serum, or at least 10% serum. In some embodiments the media comprises less than or equal to 0.1% serum, less than or equal to 0.2% serum, less than or equal to 0.3% serum, less than or equal to 0.4% serum, less than or equal to 0.5% serum, less than or equal to 0.6% serum, less than or equal to 0.7% serum, less than or equal to 0.8% serum, less than or equal to 0.9% serum, less than or equal to 1% serum, less than or equal to 2% serum, less than or equal to 3% serum, less than or equal to 4% serum, less than or equal to 5% serum, less than or equal to 6% serum, less than or equal to 7% serum, less than or equal to 8% serum, less than or equal to 9% serum, or less than or equal to 10% serum.

### D. Methods of Culturing Primary Mammalian Cells

The inventions disclosed herein are based, in part, on the inventors' surprising discovery that primary mammalian cells may be maintained in culture at a temperature of at least 12° C. (such as at least 13° C., at least 14° C., at least 15° C., at least 16° C., at least 17° C., or at least 18° C.), but below the body temperature of the mammal the cells are derived from, while retaining the viability and high functional performance potential of the primary mammalian cells. This result is surprising because primary cells are fragile and the art has generally considered special care necessary to maintain primary cells that retain the ability to achieve and maintain a state of high functional competency.

In some embodiments the primary mammalian cells are maintained at a temperature of from 12° C. to 35° C., from 12° C. to 30° C., from 12° C. to 25° C., from 12° C. to 20° C., from 12° C. to 15° C., from 15° C. to 35° C., from 15° C. to 30° C., from 15° C. to 25° C., from 15° C. to 20° C., from 18° C. to 35° C., from 18° C. to 30° C., from 18° C. to 25° C., from 18° C. to 20° C., from 20° C. to 35° C., from 20° C. to 30° C., from 20° C. to 25° C., from 25° C. to 35° C., from 25° C. to 30° C., or from 30° C. to 35° C. for a first culture period, while retaining the viability and high functional performance potential of the primary mammalian cells.

In some embodiments the primary mammalian cells are maintained at a temperature of no more than 35° C., no more than 34° C., no more than 32° C., no more than 30° C., no more than 28° C., no more than 26° C., no more than 24° C., no more than 22° C., no more than 20° C., no more than 18° C., no more than 16° C., no more than 14° C., or no more than 12° C., for a first culture period, while retaining the viability and high functional performance potential of the primary mammalian cells.

In some embodiments the first culture period is from 1 to 24 hours. In some embodiments the first culture period is from 2 to 24 hours, from 4 to 24 hours, from 6 to 24 hours, from 8 to 24 hours, from 10 to 24 hours, from 12 to 24 hours, from 14 to 24 hours, from 16 to 24 hours, from 18 to 24 hours, from 20 to 24 hours, or from 2 to 24 hours. In some embodiments the first culture period is from 2 to 20 hours, from 4 to 20 hours, from 6 to 20 hours, from 8 to 20 hours, from 10 to 20 hours, from 12 to 20 hours, from 14 to 20 hours, from 16 to 20 hours, or from 18 to 20 hours. In some embodiments the first culture period is from 2 to 16 hours, from 4 to 16 hours, from 6 to 16 hours, from 8 to 16 hours, from 10 to 16 hours, from 12 to 16 hours, or from 14 to 16 hours. In some embodiments the first culture period is from 2 to 12 hours, from 4 to 12 hours, from 6 to 12 hours, from 8 to 12 hours, or from 10 to 12 hours. In some embodiments the first culture period is from 2 to 10 hours, from 4 to 10 hours, from 6 to 10 hours, or from 8 to 10 hours. In some embodiments the first culture period is from 2 to 8 hours, from 4 to 8 hours, or from 6 to 8 hours. In some embodiments the first culture period is from 2 to 6 hours or from 4 to 6. In some embodiments the first culture period is from 2 to 4 hours.

In some embodiments the first culture period is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 14 hours, at least 16 hours, at least 18 hours, at least 20 hours, at least 22 hours, or at least 24 hours.

In some embodiments, during the first culture period the primary cells are maintained for one or more periods of time at a culture temperature at or below a certain temperature threshold. Thus, for example, in some embodiments the primary mammalian cells are maintained for a first culture period at a temperature of no more than X ° C., and for a subperiod during the first culture period the primary mammalian cells are maintained at a temperature of no more than Y ° C., wherein Y is less than X. In some embodiments Y is 1° C. less than X, 2° C. less than X, 3° C. less than X, 4° C. less than X, 5° C. less than X, 6° C. less than X, 1° C. less than X, 7° C. less than X, 8° C. less than X, 9° C. less than X, 10° C. less than X, 11° C. less than X, 12° C. less than X, 13° C. less than X, 14° C. less than X, or 15° C. less than X. The subperiod during the first culture period may be from 1 to 12 hours, from 1 to 10

In some embodiments the subperiod during the first culture period is from 2 to 16 hours, from 4 to 16 hours, from 6 to 16 hours, from 8 to 16 hours, from 10 to 16 hours, from 12 to 16 hours, or from 14 to 16 hours. In some embodiments the subperiod during the first culture period is from 2 to 12 hours, from 4 to 12 hours, from 6 to 12 hours, from 8 to 12 hours, or from 10 to 12 hours. In some embodiments the subperiod during the first culture period is from 2 to 10 hours, from 4 to 10 hours, from 6 to 10 hours, or from 8 to 10 hours. In some embodiments the subperiod during the first culture period is from 2 to 8 hours, from 4 to 8 hours, or from 6 to 8 hours. In some embodiments the subperiod during the first culture period is from 2 to 6 hours or from 4 to 6. In some embodiments the subperiod during the first culture period is from 2 to 4 hours. In some embodiments the subperiod during the first culture period is from 1 to 2 hours. In some embodiments the subperiod during the first culture period is at least 1, 2, 3, 4, 5, or 6 hours.

Another unique feature of the first culture period compared to standard methods of culturing primary mammalian cells is the range of temperature of the cell culture during the first culture period. In some embodiments the temperature of the cell culture varies over a range of at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. during the first culture period. In some embodiments the temperature of the cell culture varies over a range of from 2° C. to 16° C., from 2° C. to 14° C., from 2° C. to 12° C., from 2° C. to 10° C., from 2° C. to 8° C., from 2° C. to 6° C., from 2° C. to 4° C., 4° C. to 16° C., from 4° C. to 14° C., from 4° C. to 12° C., from 4° C. to 10° C., from 4° C. to 8° C., from 4° C. to 6° C., 6° C. to 16° C., from 6° C. to 14° C., from 6° C. to 12° C., from 6° C. to 10° C., from 6° C. to 8° C., 8° C. to 16° C., from 8° C. to 14° C., from 8° C. to 12° C., from 8° C. to 10° C., 10° C. to 16° C., from 10° C. to 14° C., from 10° C. to 12° C., 12° C. to 16° C., or from 12° C. to 14° C. during the first culture period.

The methods of the invention provide primary mammalian cells at the end of the first culture period that retain the ability to achieve high functional performance when incubated at a temperature above 35° C. In some embodiments the temperature above 35° C. at or close to the range of body temperature considered normal for the mammal that the primary cells are derived from. For human primary cells the cells are typically cultured at from 36° C. to 38° C., such as at 37° C.

In some embodiments the methods of culturing primary mammalian cells comprise providing a culture comprising primary mammalian cells maintained at a temperature of no more than 35° C. for a first culture period; and incubating the culture comprising primary mammalian cells at a temperature above 35° C. for a second culture period to thereby provide a culture of primary mammalian cells with high functional performance. In some embodiments the second culture period is for from 1 to 24 hours. In some embodiments the second culture period is from 2 to 24 hours, from 4 to 24 hours, from 6 to 24 hours, from 8 to 24 hours, from 10 to 24 hours, from 12 to 24 hours, from 14 to 24 hours, from 16 to 24 hours, from 18 to 24 hours, from 20 to 24 hours, or from 2 to 24 hours. In some embodiments the second culture period is from 2 to 20 hours, from 4 to 20 hours, from 6 to 20 hours, from 8 to 20 hours, from 10 to 20 hours, from 12 to 20 hours, from 14 to 20 hours, from 16 to 20 hours, or from 18 to 20 hours. In some embodiments the second culture period is from 2 to 16 hours, from 4 to 16 hours, from 6 to 16 hours, from 8 to 16 hours, from 10 to 16 hours, from 12 to 16 hours, or from 14 to 16 hours. In some embodiments the second culture period is from 2 to 12 hours, from 4 to 12 hours, from 6 to 12 hours, from 8 to 12 hours, or from 10 to 12 hours. In some embodiments the second culture period is from 2 to 10 hours, from 4 to 10 hours, from 6 to 10 hours, or from 8 to 10 hours. In some embodiments the second culture period is from 2 to 8 hours, from 4 to 8 hours, or from 6 to 8 hours. In some embodiments the second culture period is from 2 to 6 hours or from 4 to 6. In some embodiments the second culture period is from 2 to 4 hours. In some embodiments the second culture period is from 1 to 5 days, from 1 to 4 days, from 1 to 3 days, or from 1 to 2 days.

In some embodiments, following the first culture period a substantial proportion of the primary cells in a culture are adhered to a culture substrate. Maintenance of adherence reflects that the functional state and health of the cells is intact. In some embodiments at least 70%, at least 75%, at least 80%, at least 85%, at least 90%, at least 95%, at least 96%, at least 97%, at least 98%, at least 99%, or at least 99.5% of the primary cells in a culture or co-culture remain adhered to a culture substrate following a first culture period. In some embodiments the primary cells are hepatocytes. In some embodiments the culture is a hepatocyte-stromal cell coculture.

In some embodiments that utilize primary mammalian hepatocytes as a primary mammalian cell type in a culture, the level of metabolic activity of the cultured primary hepatocytes is retained following a first culture period, or a first and second culture period. In some embodiments the second culture period comprises culture at the optimal culture temperature for the primary cell type (e.g., 37 C for human primary cells).

In some embodiments the metabolic function of the hepatocyte-stromal cell coculture is long enduring following the first culture period, or following the first and second culture periods. In some embodiments the second culture period is for at least one day, at least two days, at least three days, at least five days, at least seven days, at least ten days, at least fourteen days, at least twenty-one days, or at least twenty-eight days. In some embodiments the metabolic function of the hepatocyte-stromal cell coculture is determined by measuring an activity selected from gene expression, cell function, metabolic activity, morphology, and a combination thereof, of the hepatocytes in the coculture. In some embodiments the metabolic function of the hepatocyte-stromal cell coculture is determined by measuring the level of expression and/or activity of at least one CYP450 enzyme. The level of expression and/or activity of at least one CYP450 enzyme may be measured by measuring expression of the CYP450 enzyme mRNA, by measuring expression of the CYP450 enzyme protein, or by a functional assay of CYP450 enzyme activity. In some embodiments, the metabolic activity is a CYP450 enzyme activity. In some embodiments, the CYP450 enzyme is a CYP450 enzyme selected from CYP1A2, CYP1B1, CYP2A6, CYP2B6, CYP2C, CYP2D6, CYP2E1, CYP2F1, CYP2J2, CYP3A4, CYP4A, and CYP4B.

In some embodiments the metabolic function of primary hepatocytes in culture endures during the second culture period for at least seven days. In some embodiments the metabolic function of primary hepatocytes in culture endures during the second culture period for at least fourteen days. In some embodiments the metabolic function of primary hepatocytes in culture endures during the second culture period for at least twenty-one days. In some embodiments the metabolic function of primary hepatocytes in culture endures during the second culture period for at least twenty-eight days.

In some embodiments the culture comprising primary mammalian cells is cultured in serum-free or essentially serum-free media during the first and/or second culture period. In some embodiments the culture comprising primary mammalian cells is cultured in media containing serum during the first and/or second culture period. In some embodiments the media comprises 0.1% serum, 0.2% serum, 0.3% serum, 0.4% serum, 0.5% serum, 0.6% serum, 0.7% serum, 0.8% serum, 0.9% serum, 1% serum, 2% serum, 3% serum, 4% serum, 5% serum, 6% serum, 7% serum, 8% serum, 9% serum, or 10% serum. In some embodiments the media comprises at least 0.1% serum, at least 0.2% serum, at least 0.3% serum, at least 0.4% serum, at least 0.5% serum, at least 0.6% serum, at least 0.7% serum, at least 0.8% serum, at least 0.9% serum, at least 1% serum, at least 2% serum, at least 3% serum, at least 4% serum, at least 5% serum, at least 6% serum, at least 7% serum, at least 8% serum, at least 9% serum, or at least 10% serum. In some embodiments the media comprises less than or equal to 0.1% serum, less than or equal to 0.2% serum, less than or equal to 0.3% serum, less than or equal to 0.4% serum, less than or equal to 0.5% serum, less than or equal to 0.6% serum, less than or equal to 0.7% serum, less than or equal to 0.8% serum, less than or equal to 0.9% serum, less than or equal to 1% serum, less than or equal to 2% serum, less than or equal to 3% serum, less than or equal to 4% serum, less than or equal to 5% serum, less than or equal to 6% serum, less than or equal to 7% serum, less than or equal to 8% serum, less than or equal to 9% serum, or less than or equal to 10% serum.

### E. Methods of Shipping Cultured Primary Mammalian Cells

This invention also encompasses methods of shipping cultures comprising primary mammalian cells. The culture comprising primary mammalian cells may be any culture of primary mammalian cells of the invention. The methods typically comprise:

A) providing a cell culture microplate comprising a cell culture comprising primary mammalian cells;

B) incorporating the cell culture microplate of A into a system for culturing cells, the manufacture comprising: a first container comprising interlocking upper and lower portions; a second container comprising interlocking upper and lower portions, the first container disposed within the second container; and a heat source disposed within the second container and outside of the first container; wherein the cell culture microplate is placed within the first container; and

C) transporting the system containing the cell culture microplate by land and/or sea from an origin to a destination; wherein the cell culture microplate is maintained at a temperature of no more than 38° C. while in transit from the origin to the destination.

In some embodiments the methods further comprise seeding primary mammalian cells onto a culture surface of at least one well of the culture microplate to provide the cell culture microplate comprising a cell culture comprising primary mammalian cells of A.

In some embodiments, upon arrival at the destination the culture of primary mammalian cells retains the ability to achieve high functional performance when incubated at the optimum incubation temperature for the primary cells.

The system used in the method may be any system disclosed herein.

The cell culture comprising primary mammalian cells may be any cell culture disclosed herein.

In some embodiments, during the transporting the primary mammalian cells are maintained at a temperature of from 10° C. to 35° C., from 10° C. to 30° C., from 10° C. to 25° C., from 10° C. to 20° C., from 10° C. to 15° C., from 15° C. to 35° C., from 15° C. to 30° C., from 15° C. to 25° C., from 15° C. to 20° C., from 20° C. to 35° C., from 20° C. to 30° C., from 20° C. to 25° C., from 25° C. to 35° C., from 25° C. to 30° C., or from 30° C. to 35° C. for a first culture period, while retaining the viability and high functional performance potential of the primary mammalian cells.

In some embodiments, during the transporting the primary mammalian cells are maintained at a temperature of no more than 35° C., no more than 34° C., no more than 32° C., no more than 30° C., no more than 28° C., no more than 26° C., no more than 24° C., no more than 22° C., no more than 20° C., no more than 18° C., no more than 16° C., no more than 14° C., or no more than 12° C., for a first culture period, while retaining the viability and high functional performance potential of the primary mammalian cells.

In some embodiments, during the transporting the first culture period is from 1 to 24 hours. In some embodiments the first culture period is from 2 to 24 hours, from 4 to 24 hours, from 6 to 24 hours, from 8 to 24 hours, from 10 to 24 hours, from 12 to 24 hours, from 14 to 24 hours, from 16 to 24 hours, from 18 to 24 hours, from 20 to 24 hours, or from 2 to 24 hours. In some embodiments the first culture period is from 2 to 20 hours, from 4 to 20 hours, from 6 to 20 hours, from 8 to 20 hours, from 10 to 20 hours, from 12 to 20 hours, from 14 to 20 hours, from 16 to 20 hours, or from 18 to 20 hours. In some embodiments the first culture period is from 2 to 16 hours, from 4 to 16 hours, from 6 to 16 hours, from 8 to 16 hours, from 10 to 16 hours, from 12 to 16 hours, or from 14 to 16 hours. In some embodiments the first culture period is from 2 to 12 hours, from 4 to 12 hours, from 6 to 12 hours, from 8 to 12 hours, or from 10 to 12 hours. In some embodiments the first culture period is from 2 to 10 hours, from 4 to 10 hours, from 6 to 10 hours, or from 8 to 10 hours. In some embodiments the first culture period is from 2 to 8 hours, from 4 to 8 hours, or from 6 to 8 hours. In some embodiments the first culture period is from 2 to 6 hours or from 4 to 6. In some embodiments the first culture period is from 2 to 4 hours.

In some embodiments, during the transporting the first culture period is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 14 hours, at least 16 hours, at least 18 hours, at least 20 hours, at least 22 hours, or at least 24 hours.

In some embodiments, during the transporting, during the first culture period the primary cells are maintained for one or more periods of time at a culture temperature at or below a certain temperature threshold. Thus, for example, in some embodiments the primary mammalian cells are maintained for a first culture period at a temperature of no more than X ° C., and for a subperiod during the first culture period the primary mammalian cells are maintained at a temperature of no more than Y ° C., wherein Y is less than X. In some embodiments Y is 1° C. less than X, 2° C. less than X, 3° C. less than X, 4° C. less than X, 5° C. less than X, 6° C. less than X, 1° C. less than X, 7° C. less than X, 8° C. less than X, 9° C. less than X, 10° C. less than X, 11° C. less than X, 12° C. less than X, 13° C. less than X, 14° C. less than X, or 15° C. less than X. The subperiod during the first culture period may be from 1 to 12 hours, from 1 to 10

In some embodiments the subperiod during the first culture period is from 2 to 16 hours, from 4 to 16 hours, from 6 to 16 hours, from 8 to 16 hours, from 10 to 16 hours, from 12 to 16 hours, or from 14 to 16 hours. In some embodiments the subperiod during the first culture period is from 2 to 12 hours, from 4 to 12 hours, from 6 to 12 hours, from 8 to 12 hours, or from 10 to 12 hours. In some embodiments the subperiod during the first culture period is from 2 to 10 hours, from 4 to 10 hours, from 6 to 10 hours, or from 8 to 10 hours. In some embodiments the subperiod during the first culture period is from 2 to 8 hours, from 4 to 8 hours, or from 6 to 8 hours. In some embodiments the subperiod during the first culture period is from 2 to 6 hours or from 4 to 6. In some embodiments the subperiod during the first culture period is from 2 to 4 hours. In some embodiments the subperiod during the first culture period is from 1 to 2 hours. In some embodiments the subperiod during the first culture period is at least 1, 2, 3, 4, 5, or 6 hours.

Another unique feature of the first culture period compared to standard methods of transporting primary mammalian cells is the range of temperature of the cell culture during the first culture period. In some embodiments the temperature of the cell culture varies over a range of at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., or at least 15° C. during the first culture period. In some embodiments the temperature of the cell culture varies over a range of from 2° C. to 16° C., from 2° C. to 14° C., from 2° C. to 12° C., from 2° C. to 10° C., from 2° C. to 8° C., from 2° C. to 6° C., from 2° C. to 4° C., 4° C. to 16° C., from 4° C. to 14° C., from 4° C. to 12° C., from 4° C. to 10° C., from 4° C. to 8° C., from 4° C. to 6° C., 6° C. to 16° C., from 6° C. to 14° C., from 6° C. to 12° C., from 6° C. to 10° C., from 6° C. to 8° C., 8° C. to 16° C., from 8° C. to 14° C., from 8° C. to 12° C., from 8° C. to 10° C., 10° C. to 16° C., from 10° C. to 14° C., from 10° C. to 12° C., 12° C. to 16° C., or from 12° C. to 14° C. during the first culture period.

The methods of the invention provide primary mammalian cells at the end of the transporting that retain the ability to achieve high functional performance when incubated at a temperature above 35° C. In some embodiments the temperature above 35° C. at or close to the range of body temperature considered normal for the mammal that the primary cells are derived from. For human primary cells the cells are typically cultured at from 36° C. to 38° C., such as at 37° C.

In some embodiments, after the transporting the methods further comprise providing a culture comprising primary mammalian cells maintained at a temperature of no more than 35° C. for a first culture period; and incubating the culture comprising primary mammalian cells at a temperature above 35° C. for a second culture period to thereby provide a culture of primary mammalian cells with high functional performance. In some embodiments the second culture period is for from 1 to 24 hours. In some embodiments the second culture period is from 2 to 24 hours, from 4 to 24 hours, from 6 to 24 hours, from 8 to 24 hours, from 10 to 24 hours, from 12 to 24 hours, from 14 to 24 hours, from 16 to 24 hours, from 18 to 24 hours, from 20 to 24 hours, or from 2 to 24 hours. In some embodiments the second culture period is from 2 to 20 hours, from 4 to 20 hours, from 6 to 20 hours, from 8 to 20 hours, from 10 to 20 hours, from 12 to 20 hours, from 14 to 20 hours, from 16 to 20 hours, or from 18 to 20 hours. In some embodiments the second culture period is from 2 to 16 hours, from 4 to 16 hours, from 6 to 16 hours, from 8 to 16 hours, from 10 to 16 hours, from 12 to 16 hours, or from 14 to 16 hours. In some embodiments the second culture period is from 2 to 12 hours, from 4 to 12 hours, from 6 to 12 hours, from 8 to 12 hours, or from 10 to 12 hours. In some embodiments the second culture period is from 2 to 10 hours, from 4 to 10 hours, from 6 to 10 hours, or from 8 to 10 hours. In some embodiments the second culture period is from 2 to 8 hours, from 4 to 8 hours, or from 6 to 8 hours. In some embodiments the second culture period is from 2 to 6 hours or from 4 to 6. In some embodiments the second culture period is from 2 to 4 hours. In some embodiments the second culture period is from 1 to 5 days, from 1 to 4 days, from 1 to 3 days, or from 1 to 2 days.

In some embodiments, following the first culture period a substantial proportion of the primary cells in a culture are adhered to a culture substrate. Maintenance of adherence reflects that the functional state and health of the cells is intact. In some embodiments at least 70%, at least 75%, at least 80%, at least 85%, at least 90%, at least 95%, at least 96%, at least 97%, at least 98%, at least 99%, or at least 99.5% of the primary cells in a culture or co-culture remain adhered to a culture substrate following a first culture period. In some embodiments the primary cells are hepatocytes. In some embodiments the culture is a hepatocyte-stromal cell coculture.

In some embodiments that utilize primary mammalian hepatocytes as a primary mammalian cell type in a culture, the level of metabolic activity of the cultured primary hepatocytes is retained following a first culture period, or a first and second culture period. In some embodiments the second culture period comprises culture at the optimal culture temperature for the primary cell type (e.g., 37 C for human primary cells).

In some embodiments the metabolic function of the hepatocyte-stromal cell coculture is long enduring following the first culture period, or following the first and second culture periods. In some embodiments the second culture period is for at least one day, at least two days, at least three days, at least five days, at least seven days, at least ten days, at least fourteen days, at least twenty-one days, or at least twenty-eight days. In some embodiments the metabolic function of the hepatocyte-stromal cell coculture is determined by measuring an activity selected from gene expression, cell function, metabolic activity, morphology, and a combination thereof, of the hepatocytes in the coculture. In some embodiments the metabolic function of the hepatocyte-stromal cell coculture is determined by measuring the level of expression and/or activity of at least one CYP450 enzyme. The level of expression and/or activity of at least one CYP450 enzyme may be measured by measuring expression of the CYP450 enzyme mRNA, by measuring expression of the CYP450 enzyme protein, or by a functional assay of CYP450 enzyme activity. In some embodiments, the metabolic activity is a CYP450 enzyme activity. In some embodiments, the CYP450 enzyme is a CYP450 enzyme selected from CYP1A2, CYP1B1, CYP2A6, CYP2B6, CYP2C, CYP2D6, CYP2E1, CYP2F1, CYP2J2, CYP3A4, CYP4A, and CYP4B.

In some embodiments the metabolic function of primary hepatocytes in culture endures during the second culture period for at least seven days. In some embodiments the metabolic function of primary hepatocytes in culture endures during the second culture period for at least fourteen days. In some embodiments the metabolic function of primary hepatocytes in culture endures during the second culture period for at least twenty-one days. In some embodiments the metabolic function of primary hepatocytes in culture endures during the second culture period for at least twenty-eight days.

In some embodiments the culture comprising primary mammalian cells is cultured in serum-free or essentially serum-free media during the first and/or second culture period. In some embodiments the culture comprising primary mammalian cells is cultured in media containing serum during the first and/or second culture period. In some embodiments the media comprises 0.1% serum, 0.2% serum, 0.3% serum, 0.4% serum, 0.5% serum, 0.6% serum, 0.7% serum, 0.8% serum, 0.9% serum, 1% serum, 2% serum, 3% serum, 4% serum, 5% serum, 6% serum, 7% serum, 8% serum, 9% serum, or 10% serum. In some embodiments the media comprises at least 0.1% serum, at least 0.2% serum, at least 0.3% serum, at least 0.4% serum, at least 0.5% serum, at least 0.6% serum, at least 0.7% serum, at least 0.8% serum, at least 0.9% serum, at least 1% serum, at least 2% serum, at least 3% serum, at least 4% serum, at least 5% serum, at least 6% serum, at least 7% serum, at least 8% serum, at least 9% serum, or at least 10% serum. In some embodiments the media comprises less than or equal to 0.1% serum, less than or equal to 0.2% serum, less than or equal to 0.3% serum, less than or equal to 0.4% serum, less than or equal to 0.5% serum, less than or equal to 0.6% serum, less than or equal to 0.7% serum, less than or equal to 0.8% serum, less than or equal to 0.9% serum, less than or equal to 1% serum, less than or equal to 2% serum, less than or equal to 3% serum, less than or equal to 4% serum, less than or equal to 5% serum, less than or equal to 6% serum, less than or equal to 7% serum, less than or equal to 8% serum, less than or equal to 9% serum, or less than or equal to 10% serum.

### Examples

### Example 1: Embodiment of System of the Invention

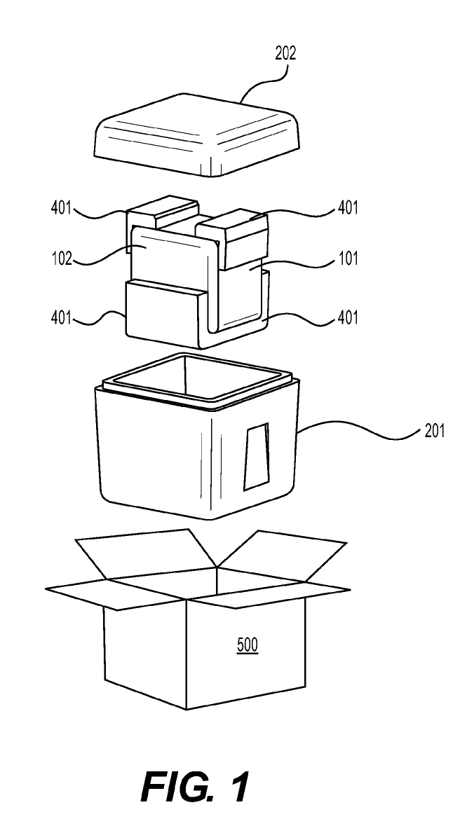

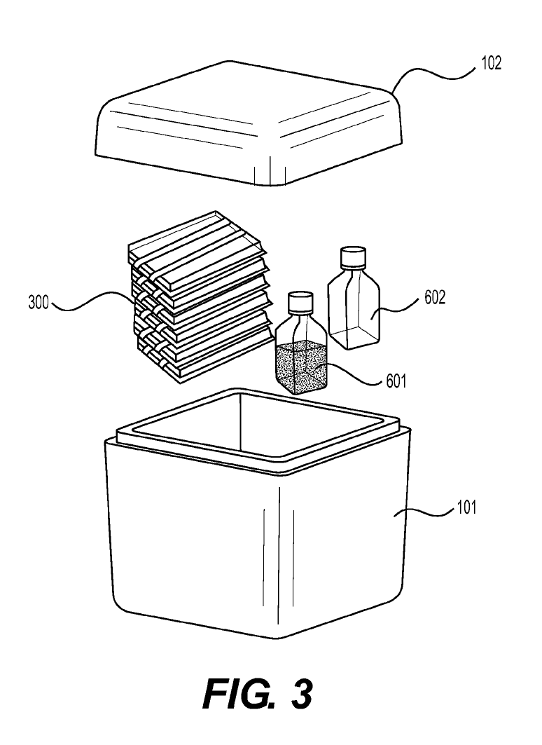

A system was designed for shipping cultures of primary mammalian cells. In designing the manufacture the goal was to maintain the temperature of a cell culture plate at from 12° C. to 38° C. The manufacture is designed to maintain the temperature of a cell culture plate for a period of at least 30 hours, independently of the ambient temperature during shipment. The manufacture has a container inside a container configuration shown in FIGS. 1-3. In addition, it incorporates chemical heat packs as a source of heat energy to maintain the temperature of the cell culture plate above 12° C. The system comprises a first container (100) comprising interlocking upper (101) and lower (102) portions; a second container (200) comprising interlocking upper (201) and lower (202) portions, the first container (100) disposed within the second container (200); a cell culture microplate (300) disposed within the first container; two chemical heat packs (410) disposed within the second container and outside of the first container; and four stabilizers (401) comprising a first edge adjacent to an outer wall of the first chamber and a second edge adjacent to an inner wall of the second chamber. Each component of the manufacture used in the subsequent examples in described more fully below.

First Container:

The first container (100) is also made of 1.5 inch thick EPS. The inside dimensions of the first container are 8 inches long, 6 inches wide, and 7 inches high. The dimensions of the first container provide for an air gap between the first and second containers when the first container is placed inside the second. The air gap facilitates functioning of the heat packs and circulation of heat generated by the heat packs. The purpose of the first container two fold. First, it isolates the interior of the first container from the second container to limit the rate of temperature variation over time inside the first container. Second, the interior provides a semi-controlled environment for maintaining a cell culture plate comprising a culture comprising primary mammalian cells over a culture period. The first container also comprises upper (102) and lower (101) portions which interlock. This arrangement allows a cell culture plate to be easily placed inside and later removed from the first container. The brims of the lower portion and the upper portion have a matched locking grove structure which not only provides tight sealing but also reduces heat transfer.

Second Container:

The second container (200) is made of Extruded Polystyrene (EPS) foam. The thickness of the walls of the second container is 1.5 inches. The inside dimensions of the second container are 12 inches long, 12 inches wide, and 13 inches high. The purpose of the second container is two fold. First, it isolates the inside of the container from ambient conditions outside the container and reduces the effect of ambient conditions on the temperature inside the chamber over the time course of a culture period. Second, it retains heat generated by heat packs inside the second container. The second container comprises upper (202) and lower (201) portions which interlock. This arrangement allows a first container to be placed inside the second container. The brims of the lower portion and the upper portion have a matched locking grove structure which not only provides tight sealing but also reduces the loss of heat from the second container.

Stabilizers:

The container inside container configuration in conjunction with use of heat packs maintains the internal temperature of the inner, first container at from 12° C. to 38° C. Stabilizers (401) were used to fix the location of the first container (100) inside the second container (200). This forms an air gap between the outer surface of the first container and the inner surface of the second container. Air present inside the air gap facilitates functioning of the heat packs. The stabilizers decrease the volume of the air gap between the two containers, affecting both peak temperature of the heat packs and the duration of time over which they provide the rated temperature. The stabilizers are carefully designed to fix the first container in place within the second container without significantly reducing the air gap between the two containers while limiting the surface area of the outer surface of the inner first container that is in contact with the stabilizer, thus maximizing transfer of heat from the heat packs to the inside of the first container in a uniform fashion. The placement of stabilizers is demonstrated in FIG. 1.

Heat Packs:

Heat packs are incorporated into the system and used as a source of heat energy to maintain the temperature of the cell culture plate at from 12° C. to 38° C., such as at from 18° C. to 38° C. The heat energy from the heat packs maintains the temperature inside the first container above 12° C. over 30 hours. An example is the Uniheat 40+ hours Shipping Warmer marketed by American Pioneer International LLC. The heat packs reach a surface temperature of 46° C. in 4 hours and then maintain a temperature of at least 38° C. for at least the next 40 hours.

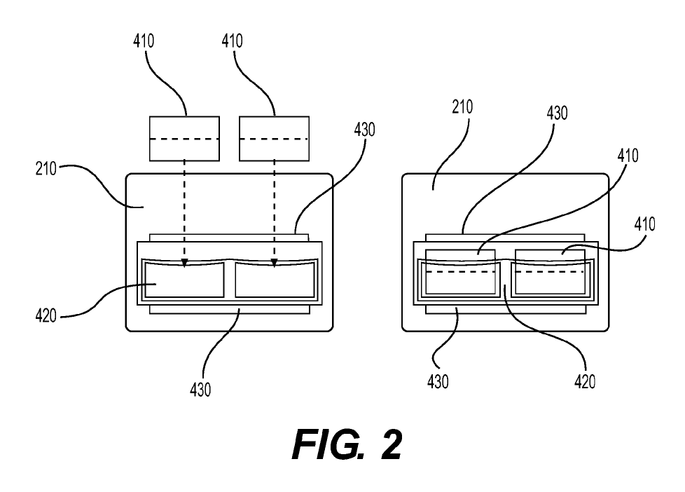

The heat packs (410) are mounted on one or more of the interior vertical sidewall(s) of the second container (210) using paper envelopes (420) secured to the inner wall(s) by tape (430). In general, from 2-12 heat packs are incorporated into the system when shipping a cell culture. The number depends on the mode of transit, weather and general transit time. The mounting of heat packs to the walls of second container is shown in FIG. 2. This configuration enables some of the heat released from the heat packs to penetrate the walls of the first container by conduction. A significant portion of the heat is also retained for a time between the two containers. This configuration thus prevents the direct propagation of heat waves from the heat packs to the interior of the first container. Therefore, the peak temperature reached in the interior of the first container is lower than the peak temperature reached in the space between the two containers. The number of heat packs required to maintain the temperature inside inner chamber from 12° C. to 38° C. (such as from 18° C. to 38° C.) depends on the temperature experienced by the system over time.

Cell Culture Plate:

Cryopreserved human hepatocytes were removed from liquid nitrogen and thawed. After thawing, cells were resuspended in medium and cell number and cell viability was determined using trypan blue exclusion. Stromal cells were passed in a CO2 incubator until used for experimental plating. On plating day cells were detached from the plate, washed, and re-suspended in medium. Cell number and viability were determined using trypan blue exclusion.

Hepatocytes and stromal cells were seeded into collagen-coated 96-well plates at a density of 30,000 hepatocytes per well. The stromal cells were growth arrested prior to seeding. Cultures were maintained for 5-6 days and expression of representative CYP450 genes was assessed. CYP450 expression was measured via mRNA content using qPCR. Cell culture media was changed and then the cell culture plate was sealed for shipping as described in Example 2.

Corrugated Box:

The sealed microplates comprising the established hepatocyte stromal cell co-cultures were then placed into the first container together with a bottle of cell culture media (601) and a bottle of dosing media (602) and the upper and lower portions of the first container joined. The first container was then placed into the second container to which chemical heat packs had been attached, and the upper and lower portions of the second container joined. The assembled second container was then placed into a corrugated box that was sealed with tape for shipment.

### Example 2: Preparation of Sealed Cell Culture Plate

A) PDMS Sheet Preparation:

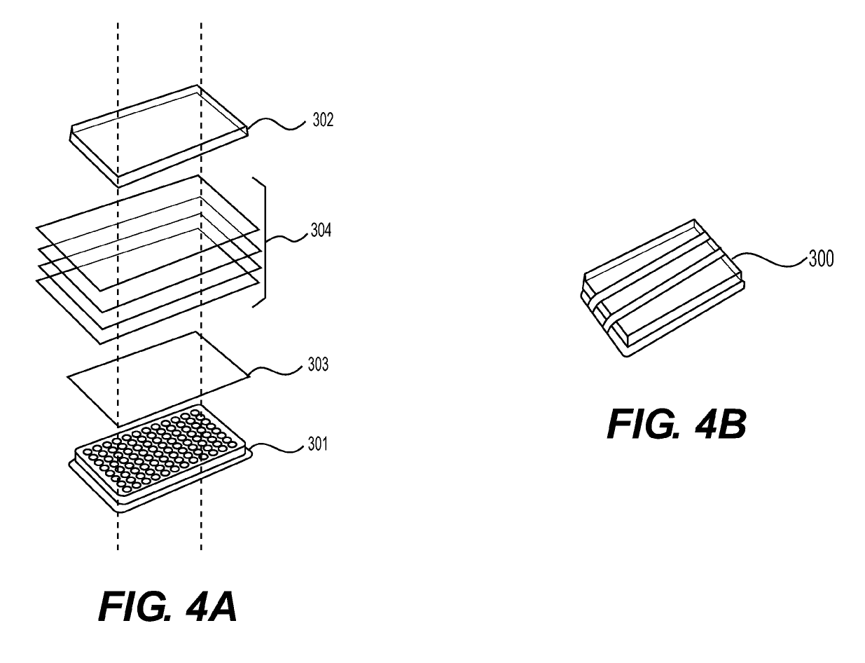

PDMS elastomer & curing agent (Sylgard 184, Dow corning, MI-48648) was mixed in a 10:1 proportion. The mixture was transferred to a glass plate with containment edges such that the dimensions of the plate are 124×79 mm (FIG. 4A). The mixture was poured such that a 2-3 mm thick layer was present on the glass plate. The mixture was allowed to spread uniformly on the glass plate, which was then placed under vacuum in a desiccator for 30 mins to remove air bubbles. A conventional oven was pre-heated to 65° C. and the glass plate was incubated in it for 2 hours. The glass plate covered with PDMS mixture was then cured overnight in conventional oven at 65° C. to ensure the complete crosslinking of PDMS polymer. The glass plate was removed from the oven and the cured PDMS sheet was removed from the glass plate and cut to 115×74 mm so that it uniformly covers a standard 96 well culture plate. PDMS sheets were either used directly or alternatively were stored at 4° C. until use. PDMS sheets were UV sterilized for 20 minutes before use.

B) Absorbent Sheet Preparation

Absorbent sheets (ZORB66, Thermosafe, USA) were cut to 156×116 mm. It is not necessary to sterilize absorbent sheets before use as they are not in direct contact with the cells.

C) Cell Packing

Human primary hepatocyte stromal cell cocultures were established in 96 well cell culture plates. Prior to packing each plate was transferred from an incubator to a hood. Culture media was aspirated off. Each well was then overfilled with pre-warmed media using a multi-pipette channel so that media fills up over the brim of each well without spilling out of it.

A UV-irradiated PDMS sheet (303) was placed on the top of the base of the plate (301) to cover the top surface of each well. Next four sheets of absorbent paper (304) were placed on top of the PDMS sheet (FIG. 4A). The plate lid (302) was then placed on top of the layered absorbent paper. Pressure was then applied to ensure formation of a tight seal. Two elastic bands (size 3.5L×¾W) were then used to hold the lid tight with the plate. The elastic bands were packed in such a way that they are parallel to each other and hold the width of the plate (FIG. 4B). This configuration ensures that each well of the culture plate is full of culture media, which ensures that the primary mammalian cells are in continuous contact with culture media during shipping.

### Example 3: Shipment of Primary Human Hepatocyte Cell Culture

A hepatocyte stromal cell co-culture was prepared and configured into a culture system as described in Examples 1 and 2. For this experiment a temperature probe was placed inside the first container to record the temperature over time. The temperature probe was a standard electronic temperature logger that records the temperature every minute. The temperature logger LIBERO Ti1-S was purchased from ELPRO Services Inc., Marietta Ohio and used as it is. It is a portable (95×40×12 mm/38 g) battery operated temperature recording device. It records the temperature of inner chamber after every minute up to 11 days.

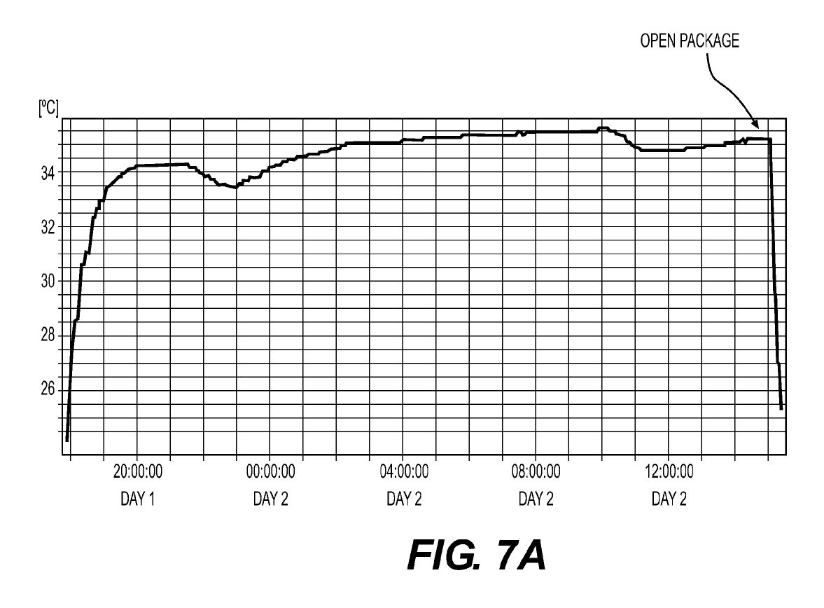

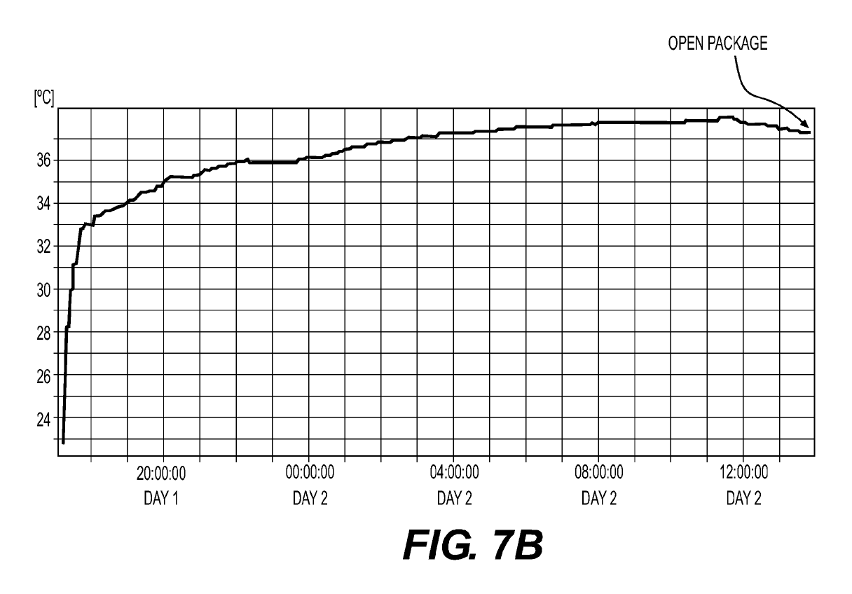

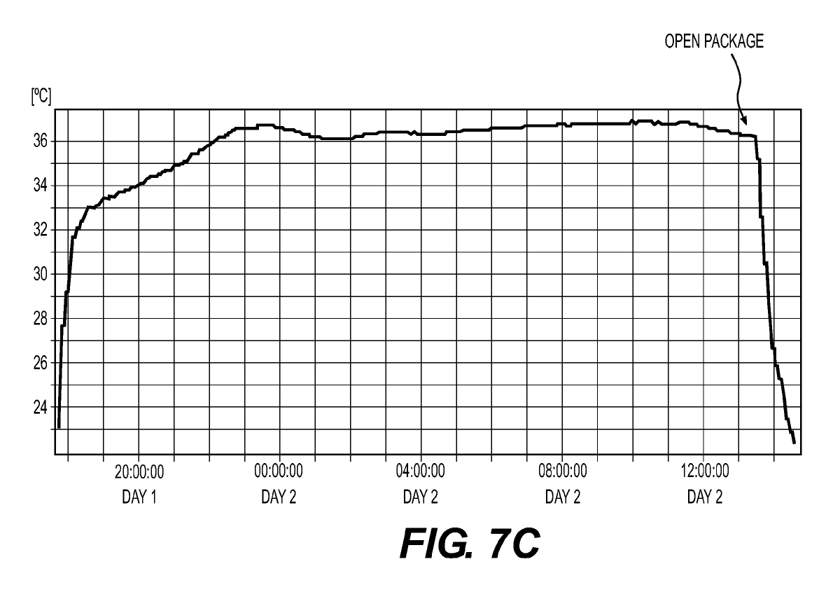

The assembled cell culture system was then shipped by courier from New Jersey to California. A graph of temperature over time is shown in FIG. 7A. Initially the temperature inside the first container is about 22° C., room temperature where the system was configured. The system was in transit by truck for about 2 hours. During that time the temperature inside the first container rapidly rose to over approximately 28.5° C. The system was then loaded onto an airplane. The airplane took off at about the 3 hour mark. At that point the system was exposed to the low ambient temperature of the airplane cargo hold. By the 5 hour mark the temperature inside the first container had dropped to approximately 20° C. As the plane reduced altitude and landed the temperature inside the first container increased to approximately 29.5° C. The system was then delivered to the destination and the containers opened. The remaining temperature recording does not represent conditions experienced by the cell culture.

After the system was opened the cell culture plate was removed, the media was changed, and the plate was incubated at 37° C. Visual inspection confirmed that substantially all of the hepatocytes in the culture remained adhered to the bottom of the wells of the cell culture plate.

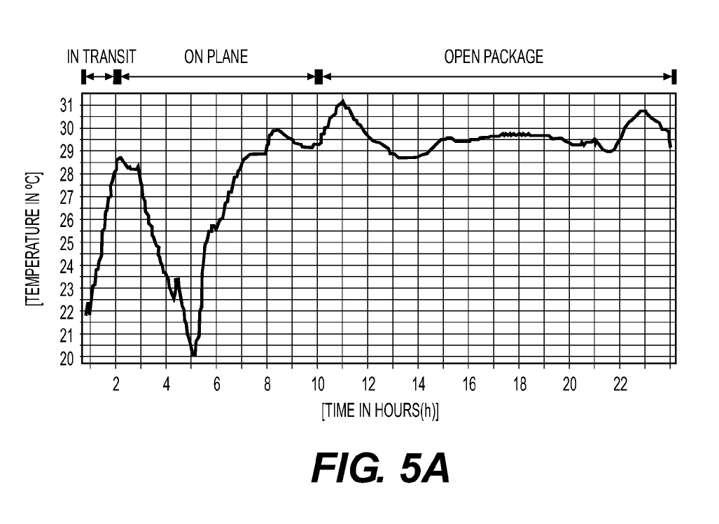

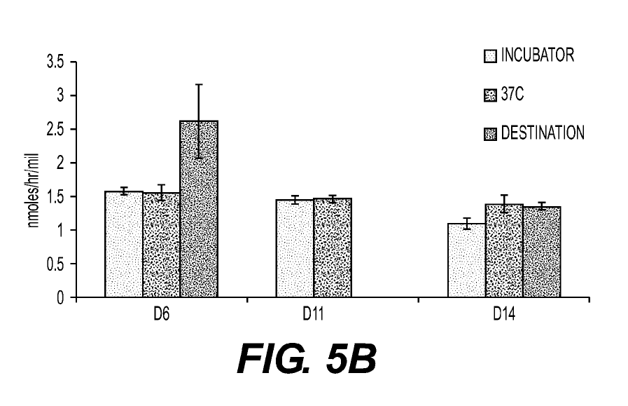

CYP450 3A4 clearance was assessed at days 6, 11, and 14. The data is presented in FIG. 5B.

This data demonstrates following shipment the primary hepatocyte-stromal cell coculture provides long-term high hepatocyte performance.

### Example 4: Shipment of Primary Human Hepatocyte Cell Culture

A hepatocyte stromal cell co-culture was prepared and configured into a culture system as described in Examples 1 and 2. For this experiment a temperature probe was placed inside the first container to record the temperature over time.

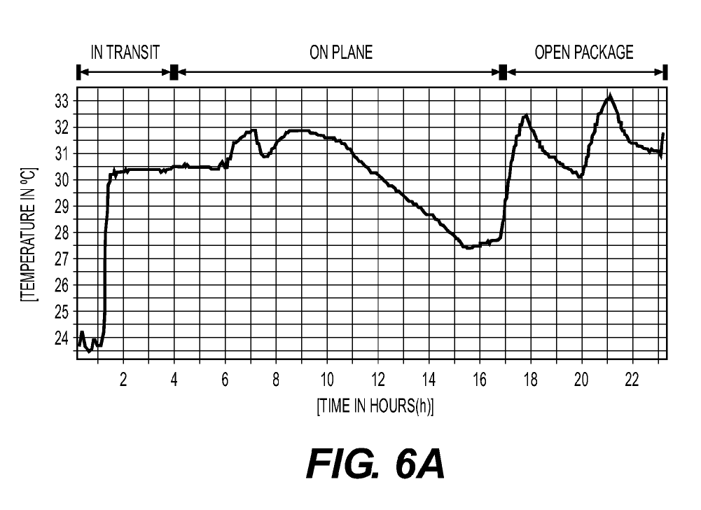

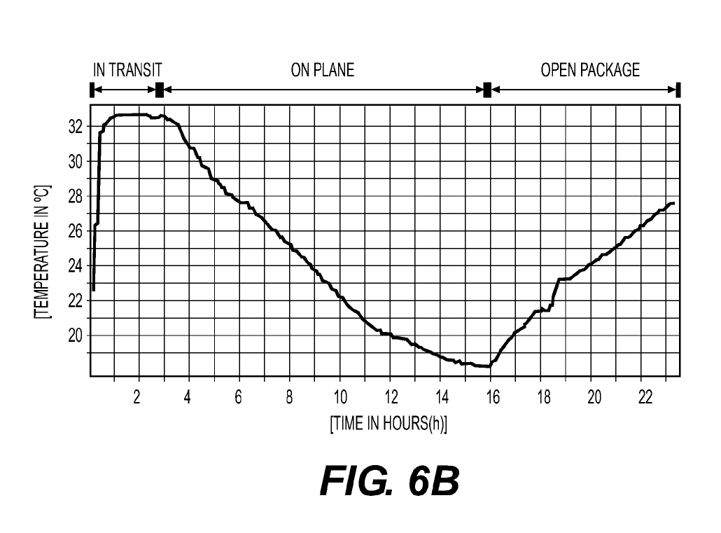

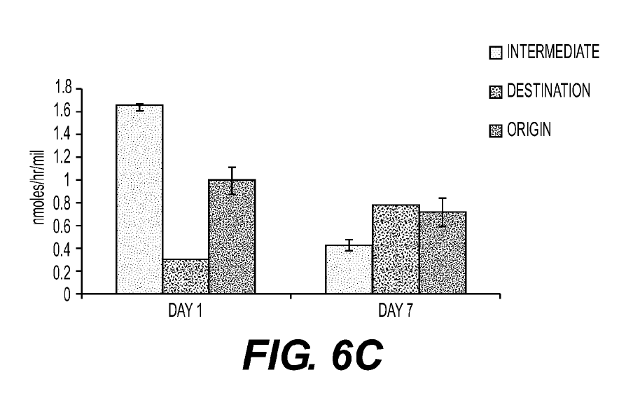

The assembled cell culture system was then shipped by courier from New Jersey to Europe. Graphs of temperature over time during this shipment are shown in FIGS. 6A and 6B.

After the system was opened the cell culture plate was removed, the media was changed, and the plate was incubated at 37° C. Visual inspection confirmed that substantially all of the hepatocytes in the culture remained adhered to the bottom of the wells of the cell culture plate.

CYP450 3A4 clearance was assessed at days 1 and 7. The data is presented in FIG. 6C.

This data demonstrates following shipment the primary hepatocyte-stromal cell coculture provides long-term high hepatocyte performance.

### Example 5: Shipment of Primary Human Hepatocyte Cell Culture

Hepatocyte stromal cell co-cultures were prepared and configured into culture systems as described in Examples 1 and 2. For this experiment a temperature probe was placed inside the first container to record the temperature over time.

The assembled cell culture systems was then shipped by courier from New Jersey to Illinois on three different shipments. Graphs of temperature over time during these shipments are shown in FIGS. 7A to 7C. Note that in this instance the shipping distance was shorter and the ambient outdoor temperature higher than for the previous experiments. This resulted in less loss of heat from the system during the shipping.

### Example 6: Electric Heater as Heat Source

The data presented in Examples 3-5 demonstrates the utility of embodiments of systems of the invention that utilize chemical heat packs as heat sources. As an alternative, the use of an electrical heating mechanism using resistive heating elements custom made from resistive nicrome wire was tested. The nicrome heating element was powered by a battery through the temperature controller. The experiments were performed to determine the size of battery required to power the heating element for maintaining the desired temperature inside the first container for 24 hours. 12V lithium ion (20 Ah @weight 10 lbs and 10 Ah @weight 5 lbs) and lead acid (9 Ah @weight 10 lbs) batteries were tested.

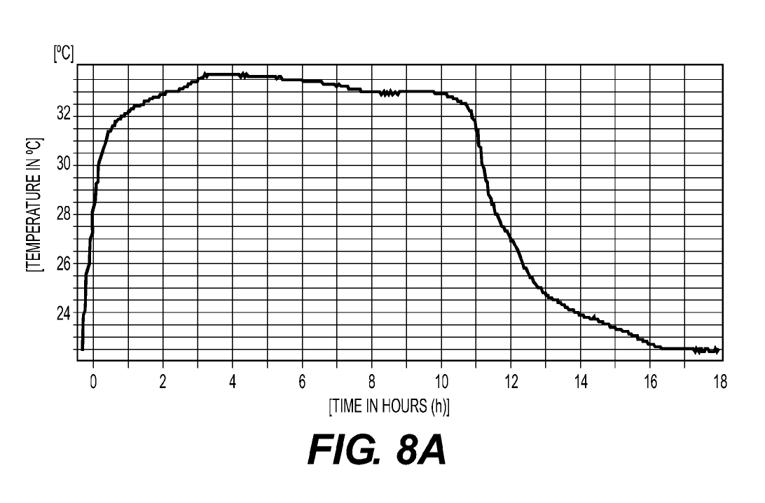

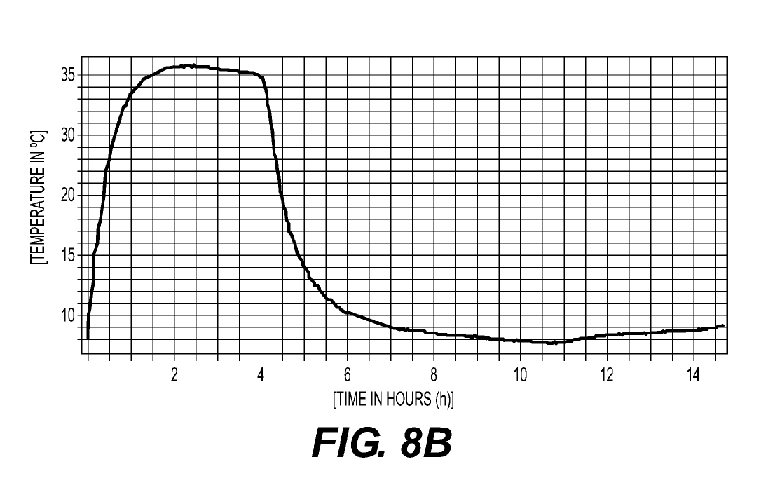

In a first experiment the electrical heating mechanism was powered using 12V 20 Ah lithium ion battery. FIG. 8A shows the temperature inside the first container of the shipping system over time when the system was subjected to room temperature conditions. The temperature inside the first container was maintained between 30° C. and 36° C. for only 11 hours and then the temperature dropped below 22° C. That suggested that the battery powered heating element was not sufficient to maintain the necessary temperature at lower ambient temperatures.

In a next experiment, conditions during shipping by airplane were mimicked by placing the system in a refrigerator. The system was maintained in the refrigerator for 24 hours. The results shown in FIG. 8B clearly show that the 12V 20 Ah lithium ion battery was only able to maintain the temperature between 18° C. to 36° C. for hours.

These results showed that battery of more than 10 lbs is necessary to power the electrical heating mechanism so as to maintain the temperature of the shipment between 18° C. to 36° C. for the necessary period of time. In addition, shipping lithium ion or lead acid battery requires explosion proof packaging which is expensive and complex. As a result, the tested battery and heating element mechanism is not appropriate for use in the systems of the invention.

### Example 7: Shipment without Heat Source

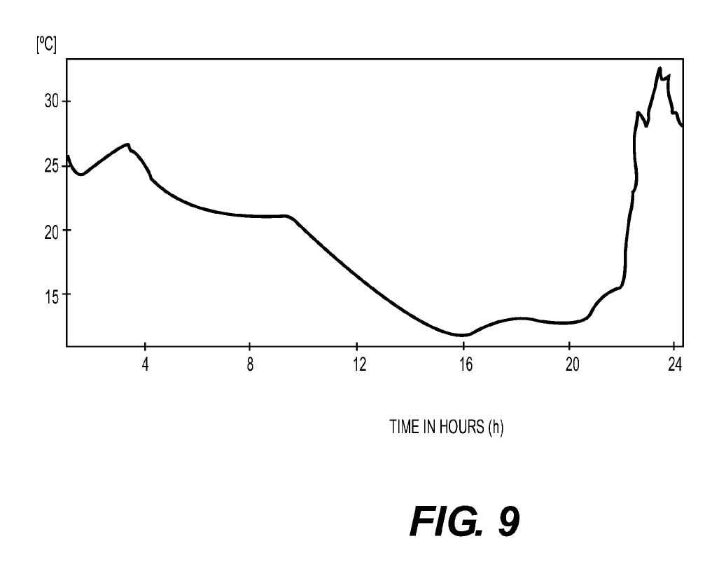

The data presented in Examples 3-5 demonstrates the utility of embodiments of systems of the invention that utilize chemical heat packs as heat sources. To assess the impact of the chemical heat packs on the temperature inside the first container a control experiment was performed in which a system similar to that used in Example 3 was configured without inclusion of a heatpack. The system was then shipped by courier from New Jersey to California. A graph of temperature over time during this shipment is shown in FIG. 9. As shown, without inclusion of a heatpack the temperature dropped to approximately 11° C. as a result of the prolonged exposure to 4° C. ambient temperature during flight. When the system was disassembled at the destination it was observed that primary hepatocytes in the culture had detached from the culture surface due to the steep drop in temperature.

It is to be understood that while various illustrative implementations have been described, the forgoing description is merely illustrative and does not limit the scope of the invention. While several examples have been provided in the present disclosure, it should be understood that the disclosed systems and methods may be embodied in many other specific forms without departing from the scope of the present disclosure.

The examples disclosed can be implemented in combinations or sub-combinations with one or more other features described herein. A variety of systems and methods may be implemented based on the disclosure and still fall within the scope of the invention. Also, the various features described or illustrated above may be combined or integrated in other systems or methods, or certain features may be omitted, or not implemented.

While various embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention.

1. A cell culture system comprising:
a first container and a second container, the first container disposed within the second container;
a cell culture plate disposed within the first container; and
a heat source disposed inside of the second container and outside of the first container.

2. (canceled)

3. The cell culture system of claim 1, wherein the first and second containers are dimensioned to provide a space between an inner wall of the second container and an outer wall of the first container.

4. (canceled)

5. The cell culture system of claim 3, wherein the heat source is positioned between the inner wall of the second container and the outer wall of the first container.

6. The cell culture system of claim 1, wherein the cell culture plate contains a culture of adherent primary mammalian cells.

7. The cell culture system of claim 6, wherein the adherent primary mammalian cells are hepatocytes.

8. The cell culture system of claim 6, wherein the cell culture plate comprises a base, a cover, and a layer of elastomeric material disposed between the base and cover to provide a liquid barrier to retain the culture media in the well.

9-10. (canceled)

11. The cell culture system of claim 1, wherein the heat source is an exothermic chemical process.

12-16. (canceled)

17. A method of making a cell culture system, comprising:
A) providing a first container, a second container, a cell culture plate, and a heat source;
B) establishing a primary mammalian cell culture in the cell culture plate; and
C) configuring the system so that
1) the first container is disposed within the second container;
2) the cell culture plate comprising the primary mammalian cell culture is disposed within the first container; and
3) the heat source is disposed inside of the second container and outside of the first container.

18-19. (canceled)

20. The method of claim 19, wherein the heat source is positioned between the inner wall of the second container and the outer wall of the first container.

21. The method of claim 17, wherein the primary mammalian cells are adhered to at least one surface of the culture plate.

22. (canceled)

23. The method of claim 22, wherein the elastomeric material is gas permeable.

24. (canceled)

25. The method of claim 17, wherein the heat source is an exothermic chemical process.

26-28. (canceled)

29. A method of culturing cells, comprising:
A) providing a cell culture system comprising a first container, a second container, a cell culture plate comprising a primary mammalian cell culture, and a heat source, wherein the cell culture system is configured such that
1) the first container is disposed within the second container;
2) the cell culture plate comprising the primary mammalian cell culture is disposed within the first container; and
3) the heat source is disposed inside of the second container and outside of the first container; and
B) maintaining the cell culture plate comprising a primary mammalian cell culture in the configured cell culture system for a first culture period.

30-31. (canceled)

32. The method of claim 31, wherein the heat source is positioned between the inner wall of the second container and the outer wall of the first container.

33. The method of claim 29, wherein the primary mammalian cells are adhered to at least one surface of the culture plate.

34-36. (canceled)

37. The method of claim 29, wherein the heat source is an exothermic chemical process.

38-42. (canceled)

43. The method of claim 29, wherein the temperature inside the first container is from 28° C. to 38° C. for the first culture period.

44. The method of claim 43, wherein the temperature inside the first container is no more than 32° C. for from one to six hours during the first culture period.

45. (canceled)

46. The method of claim 43, wherein the difference between the minimum and maximum temperature experienced by the inside of the first container during the first culture period is from 2° C. to 20° C.

47. The method of claim 29, wherein the primary cells are primary hepatocytes.

48-49. (canceled)
